(19) United States
(12) Patent Application Publication
(10) Pub. No.: US 2025/0319141 A1
(43) Pub. Date: Oct. 16, 2025

(54) USE OF MICROBIOME FOR ASSESSMENT AND TREATMENT OF OBESITY AND TYPE 2 DIABETES

(71) Applicant: The Chinese University of Hong Kong, Shatin, New Territories (CN)

(72) Inventors: Siew Chien NG, Hong Kong (CN); Ka Leung Francis CHAN, Tai Po, New Territories (CN); Zhilu XU, Sheung Shui, New Territories (CN)

(21) Appl. No.: 18/283,197

(22) PCT Filed: Mar. 31, 2022
(86) PCT No.: PCT/CN2022/084418

§ 371 Date: Sep. 20, 2023

Related U.S. Application Data
(60) Provisional application No. 63/169,481, filed on Apr. 1, 2021.

(51) Int. Cl. A61K 35/741; A61P 3/04; C12Q 1/689

(52) U.S. Cl. CPC A61K 35/741; A61P 3/04; C12Q 1/689; C12Q 2600/106

(57) ABSTRACT

The presence and quantity of certain bacterial species in a person's gastrointestinal tract is directly related to obesity and type 2 diabetes (T2D). Thus, methods are provided for reducing the risk of obesity and T2D, for treating obesity and T2D, for assessing a person's risk for obesity and T2D, as well as for determining whether obesity and T2D is microbiome-related in a subject. Also provided are kits and compositions for use in thetthese methods.

### RELATED APPLICATIONS

This application claims priority to U.S. Provisional Patent Application No. 63/169,481, filed Apr. 1, 2021, the contents of which are hereby incorporated by reference in the entirety for all purposes.

### BACKGROUND OF THE INVENTION

As living standards continue to improve globally, the number of individuals who are overweight or even obese is also rapidly increasing. Because of the serious health risks directly associated with excess body weight, this trend of an ever increasing proportion of the general population being overweight has led to a notably higher incidence of many diseases including diabetes, heart disease, hypertension, and stroke. For example, the World Health Organization (WHO) estimates that by 2030 the number of people living with diabetes will exceed 350 million worldwide. Due to the rising incidence of obesity-related diseases, their serious health implications, as well as their profound economic consequences, there exists an urgent need for new and effective means to determine individuals' risk for developing obesity and type 2 diabetes (T2D), thus permitting prophylaxis and early treatment of the individuals who are have been deemed at increased risk for obesity and T2D in an effort to ultimately reduce or eliminate their risk of later suffering from serious medical conditions associated with diabetes, hypertension, cardiovascular disease, and the like. The present invention fulfills this and other related needs by providing new methods and compositions that can effectively assess a patient's risk for obesity and T2D.

### BRIEF SUMMARY OF THE INVENTION

The invention relates to novel methods and compositions useful for assessing a subject's risk for obesity and T2D and for assessing the nature of a person's obesity and T2D-whether the diseased state is gut microbiome-dependent. In particular, the present inventors have discovered that certain microorganism species, especially certain bacteria, are present at distinctly different levels in the gastrointestinal (GI) tract of individuals depending on whether or not they are at increased risk for developing obesity and T2D. Thus, in the first aspect, the present invention provides a method for reducing the risk of obesity and type 2 diabetes (T2D) or treating obesity and T2D in a subject. The method includes the step of introducing into the subject's gastrointestinal tract an effective amount of one or more of the bacterial species selected from the group consisting of Faecalibacterium prausnitzi, Bifidobacterium longum, Eubacterium halli, Bifidobacterium bifidum, Roseburia intestinalis, Eubacterium eligens, Lachnospiraceae bacterium_5_1_63FAA, Eubacterium ventriosum, and Roseburia hominis. In some embodiments, the bacterial species does not include any one of Bifidobacterium species. In some embodiments, the bacterial species includes no more than one of Bifidobacterium species. In some embodiments, the introducing step comprises oral administration to the subject a composition comprising an effective amount of the one or more of the bacterial species. In some embodiments, the introducing step comprises delivery to the small intestine, ileum, or large intestine of the subject a composition comprising an effective amount of the one or more of the bacterial species. In some embodiments, the introducing step comprises fecal microbiota transplantation (FMT), for example by way of administering to the subject a composition comprising processed donor fecal material. In some embodiments, the processed donor fecal material is a mixture comprising fecal material taken from at least two, possibly more, lean donors, e.g., those who have BMI <23 kg/m². In some embodiments, the composition used in the method comprises no detectable amount of any species set forth in Table 2 or 4, e.g., undetectable by conventional detection methods for these specific bacterial species, such as by nucleic acid hybridization or by polymerase chain reaction (PCR). In some embodiments, the composition is orally administered. In some embodiments, the composition is directly deposited to the subject's gastrointestinal tract. In some embodiments, the level or relative abundance of the one or more of the bacterial species is determined in a first stool sample obtained from the subject prior to the introducing step and in a second stool sample obtained from the subject after the introducing step, for example, by polymerase chain reaction (PCR), preferably quantitative polymerase chain reaction (qPCR).

In a second aspect, the present invention provides a novel method for assessing an individual's risk for obesity and T2D by analyzing the profile of certain gut bacterial species. The method includes these steps: (1) determining, in a stool sample from the subject, the level or relative abundance of one or more of the bacterial species set forth in Tables 1-5; (2) determining the level or relative abundance of the same bacterial species in a stool sample from a reference cohort comprising subjects with obesity and T2D and subject without obesity and T2D; (3) generating decision trees by random forest model using data obtained from step (2) and running the level or relative abundance of one or more of the bacterial species from step (1) down the decision trees to generate a score; and (4) determining the subject with a score greater than 0.5 as having an increased risk for obesity and T2D, and determining the subject with a score no greater than 0.5 as having no increased risk for obesity and T2D.

In some embodiments, the one or more bacterial species comprise any one, any two or three bacterial species set forth in Tables 1-5. For example, the bacterial species comprises (i) Clostridium bartlettii, Haemophilus parainfluenzae, Escherichia coli, Lachnospiraceae bacterium 5_1_63FAA, Eubacterium ventriosum or, (ii) Clostridium bartlettii or, (iii) Haemophilus parainfluenzae or, (iv) Escherichia coli or, (v) Lachnospiraceae bacterium 5_1_63FAA or, (vi) Eubacterium ventriosum or, (vii) Clostridium bartlettii, Haemophilus parainfluenzae, Escherichia coli, Lachnospiraceae bacterium 5_1_63FAA or, (viii) Clostridium bartlettii, Haemophilus parainfluenzae, Escherichia coli, Eubacterium ventriosum or, (ix) Clostridium bartlettii, Haemophilus parainfluenzae, Lachnospiraceae bacterium 5_1_63FAA, Eubacterium ventriosum or, (x) Clostridium bartlettii, Escherichia coli, Lachnospiraceae bacterium 5_1_63FAA, Eubacterium ventriosum or, (xi) Haemophilus parainfluenzae, Escherichia coli, Lachnospiraceae bacterium 5_1_63FAA, Eubacterium ventriosum or, (xii) Clostridium bartlettii, Lachnospiraceae bacterium 5_1_63FAA, Eubacterium ventriosum or, (xiii) Clostridium bartlettii, Haemophilus parainfluenzae, Lachnospiraceae bacterium 5_1_63FAA or, (xiv) Haemophilus parainfluenzae, Escherichia coli, Lachnospiraceae bacterium 5_1_63FAA or, (xv) Clostridium bartlettii, Haemophilus parainfluenzae, Escherichia coli or, (xvi) Haemophilus parainfluenzae, Escherichia coli. In some embodiments, the subject has not been diagnosed with obesity. In some embodiments, the subject has not been diagnosed with T2D. In some embodiments, each of steps (1) and (2) comprises metagenomics sequencing or polymerase chain reaction (PCR), such as quantitative PCR.

In a third aspect, the present invention provides a method for assessing whether a subject has microbiome-dependent obesity and T2D. The method includes these steps: (1) determining, in a stool sample from the subject, the level or relative abundance of one or more of the bacterial species set forth in Table 5; (2) determining the level or relative abundance of the same bacterial species in a stool sample from a reference cohort comprising subjects with obesity and T2D and subject without obesity and T2D; (3) generating decision trees by random forest model using data obtained from step (2) and running the level or relative abundance of one or more of the bacterial species from step (1) down the decision trees to generate a score; and (4) determining the subject with a score greater than 0.5 as having microbiome-dependent obesity and T2D, and determining the subject with a score no greater than 0.5 as having microbiome-independent obesity and T2D.

In some embodiments, the one or more bacterial species comprise any one, any two or three bacterial species set forth in Table 5. For example, the bacterial species comprises (i) Clostridium bartlettii, Haemophilus parainfluenzae, Escherichia coli, Lachnospiraceae bacterium 5_1_63FAA, Eubacterium ventriosum or, (ii) Clostridium bartlettii or, (iii) Haemophilus parainfluenzae or, (iv) Escherichia coli or, (v) Lachnospiraceae bacterium 5_1_63FAA or, (vi) Eubacterium ventriosum or, (vii) Clostridium bartlettii, Haemophilus parainfluenzae, Escherichia coli, Lachnospiraceae bacterium 5_1_63FAA or, (viii) Clostridium bartlettii, Haemophilus parainfluenzae, Escherichia coli, Eubacterium ventriosum or, (ix) Clostridium bartlettii, Haemophilus parainfluenzae, Lachnospiraceae bacterium 5_1_63FAA, Eubacterium ventriosum or, (x) Clostridium bartlettii, Escherichia coli, Lachnospiraceae bacterium 5_1_63FAA, Eubacterium ventriosum or, (xi) Haemophilus parainfluenzae, Escherichia coli, Lachnospiraceae bacterium 5_1_63FAA, Eubacterium ventriosum or, (xii) Clostridium bartlettii, Lachnospiraceae bacterium 5_1_63FAA, Eubacterium ventriosum or, (xiii) Clostridium bartlettii, Haemophilus parainfluenzae, Lachnospiraceae bacterium 5_1_63FAA or, (xiv) Haemophilus parainfluenzae, Escherichia coli, Lachnospiraceae bacterium 5_1_63FAA or, (xv) Clostridium bartlettii, Haemophilus parainfluenzae, Escherichia coli or, (xvi) Haemophilus parainfluenzae, Escherichia coli. In some embodiments, the subject has been diagnosed with obesity. In some embodiments, the subject has been diagnosed with T2D. In some embodiments, each of steps (1) and (2) comprises metagenomics sequencing or polymerase chain reaction (PCR), such as quantitative PCR.

In a fourth aspect, the present invention provides a kit for assessing risk of obesity and type 2 diabetes (T2D) in a subject or for assessing whether a subject has microbiome-dependent obesity and T2D. The kit comprises reagents for detecting one or more of the bacterial species set forth in Tables 1-5. For example, the bacterial species comprises (i) Clostridium bartlettii, Haemophilus parainfluenzae, Escherichia coli, Lachnospiraceae bacterium 5_1_63FAA, Eubacterium ventriosum or, (ii) Clostridium bartlettii or, (iii) Haemophilus parainfluenzae or, (iv) Escherichia coli or, (v) Lachnospiraceae bacterium 5_1_63FAA or, (vi) Eubacterium ventriosum or, (vii) Clostridium bartlettii, Haemophilus parainfluenzae, Escherichia coli, Lachnospiraceae bacterium 5_1_63FAA or, (viii) Clostridium bartlettii, Haemophilus parainfluenzae, Escherichia coli, Eubacterium ventriosum or, (ix) Clostridium bartlettii, Haemophilus parainfluenzae, Lachnospiraceae bacterium 5_1_63FAA, Eubacterium ventriosum or, (x) Clostridium bartlettii, Escherichia coli, Lachnospiraceae bacterium 5_1_63FAA, Eubacterium ventriosum or, (xi) Haemophilus parainfluenzae, Escherichia coli, Lachnospiraceae bacterium 5_1_63FAA, Eubacterium ventriosum or, (xii) Clostridium bartlettii, Lachnospiraceae bacterium 5_1_63FAA, Eubacterium ventriosum or, (xiii) Clostridium bartlettii, Haemophilus parainfluenzae, Lachnospiraceae bacterium 5_1_63FAA or, (xiv) Haemophilus parainfluenzae, Escherichia coli, Lachnospiraceae bacterium 5_1_63FAA or, (xv) Clostridium bartlettii, Haemophilus parainfluenzae, Escherichia coli or, (xvi) Haemophilus parainfluenzae, Escherichia coli. In some embodiments, the kit includes two or more containers, each containing a composition comprising a reagent for polymerase chain reaction (PCR), such as quantitative PCR, for the detection of the bacterial species, such as primers and/or probes, which typically contain nucleotide sequence homologous or complementary to a polynucleotide sequence from the bacterial species.

### BRIEF DESCRIPTION OF THE DRAWINGS

FIG. 1 Differential bacterial species between subjects with both obesity and T2D (ObT2) and lean control. The green bars represent species enriched in lean control, whereas the red bars represent species enriched ObT2.

FIG. 2(A) Receiver operating characteristic (ROC) curve and the area under the curve (AUC) of the machine learning model. AUC of random forest model using: All 5 markers (red)—Clostridium bartlettii, Haemophilus parainfluenzae, Escherichia coli, Lachnospiraceae bacterium 5_1_63FAA, Eubacterium ventriosum.

FIG. 2(B) Receiver operating characteristic (ROC) curve and the area under the curve (AUC) of the machine learning model. AUC of random forest model using individual marker: Clostridium bartlettii (red), Haemophilus parainfluenzae (light blue) Escherichia coli (green), Lachnospiraceae bacterium 5_1_63FAA (dark blue); Eubacterium ventriosum (orange).

FIG. 3 Box plot depicting relative abundance of markers for machine learning model in ObT2 and lean controls.

FIG. 4(A) Risk score of a new ObT2 subject (new subject) compared to ObT2 and lean controls. FIG. 4(B) Risk score of a new lean subject (new subject) compared to ObT2 and lean controls.

FIG. 5 Impact of different FMT regimes on bodyweight loss in obese subjects. FIG. 5(A) Study diagram. In the nFMT study, recipients received 4 times of monthly mixed-donor FMT. Recipient stool samples were collected at baseline, one month since first FMT infusion, one month since last FMT and two to three months since last FMT. In the iFMT study, subject received a 3-day antibiotics preparation, followed by consecutive 5-day single-donor FMT (with a two-day interval) each week for four weeks. Patients were followed up through week 52 for clinical parameters in both studies. FIG. 5(B) Bodyweight change following FMT. Significance between studies were calculated by repeated measures ANOVA.

FIG. 6 Intensive FMT led to increased number of lean donor-derived species and resemblance to donor microbiome profile. FIG. 6(A) proportion of donor-derived species in subjects with obesity. FIG. 6(B) abundance of donor-derived species in subjects with obesity. FIG. 6(C) Bray curtis distance between microbiota in post FMT samples and respective baseline samples. FIG. 6(D) Bray Curtis distance between microbiota in recipient samples and respective donors.

FIG. 7 Mixed-donor FMT was more effective in inducing increase of Butyrate-producing bacteria compared with single-donor intensive FMT. FIG. 7(A) Heatmap depicting the abundance of butyrate-producing bacteria. FIG. 7(B) Chaol richness and Shannon diversity index of butyrate-producing bacteria in both studies were depicted. FIG. 7(C) Aggregated abundance of butyrate-producing bacteria in both studies were depicted. FIG. 7(D) Network plot depicting the association of butyrate-producing bacteria following nFMT. FIG. 7(E) Chaol richness and Shannon diversity index in both FMT regimen were depicted. Significance between studies were calculated by Wilcoxon rank sum test. Significance within same study were calculated by Wilcoxon signed-rank test.

FIG. 8 Butyrate-producing bacteria strain engraftment or replacement of in FMT recipients. FIG. 8(A) Distinct strain clusters based on SNP haplotype profile. FIG. 8(B) Strain replacement in FMT recipients at each time point. Strain clusters were defined at a tree height of 0.8 (80% dissimilarity).

FIG. 9 LDA effect size of bacterial species that significantly changed following nFMT (LDA>2, p<0.05).

FIG. 10 Lineplot depicting the relative abundance of butyrate-producing bacteria in donors and recipients following FMT and iFMT. Abundance were shown as relative abundance (%) after logarithmic transformation.

FIG. 11 Correlation of abundance change of butyrate-producing bacteria at one month after the last FMT infusion.

FIG. 12 Presence of species at baseline and 2˜3 months after last FMT infusions.

### DEFINITIONS

As used herein, the term “microbiome-dependent” describes a correlation between the presence and/or status of a physiological state (e.g., a person's body weight) or a medical condition (e.g., obesity or type 2 diabetes) and the profile of microorganisms, both in their presence and absolute or relative quantity, found in a pre-determined environment (e.g., a person's gastrointestinal tract). In the same fashion, the term “bacteriome-dependent” describes a correlation between a person's physiological/pathological condition and profile of bacterial species present in the person, such as the person's gastrointestinal tract.

“Percentage relative abundance,” when used in the context of describing the presence of a particular bacterial species (e.g., any one of those shown in any one of Tables 1-5) in relation to all bacterial species present in the same environment, refers to the relative amount of the bacterial species out of the amount of all bacterial species as expressed in a percentage form. For instance, the percentage relative abundance of one particular bacterial species can be determined by comparing the quantity of DNA specific for this species (e.g., determined by quantitative polymerase chain reaction) in one given sample with the quantity of all bacterial DNA (e.g., determined by quantitative polymerase chain reaction (PCR) and sequencing based on the 16s rRNA sequence) in the same sample.

“Absolute abundance,” when used in the context of describing the presence of a particular bacterial species (e.g., any one of those shown in Tables 1-5) in the feces, refers to the amount of DNA derived from the bacterial species out of the amount of all DNA in a fecal sample. For instance, the absolute abundance of one bacterium can be determined by comparing the quantity of DNA specific for this bacterial species (e.g., determined by quantitative PCR) in one given sample with the quantity of all fecal DNA in the same sample.

“Total bacterial load” of a fecal sample, as used herein, refers to the amount of all bacterial DNA, respectively, out of the amount of all DNA in the fecal sample. For instance, the absolute abundance of bacteria can be determined by comparing the quantity of bacteria-specific DNA (e.g., 16s rRNA determined by quantitative PCR) in one given sample with the quantity of all fecal DNA in the same sample.

The term “overweight” is used to describe a subject of excessive body weight and having a body mass index (BMI) greater than 25 (or in Asian population between 23 and 24.9). Encompassed within this term is “obese” or “obesity,” which describes a condition where the suffer has a BMI greater than 30 (or in Asian population greater than 25).

The term “treat” or “treating,” as used in this application, describes an act that leads to the elimination, reduction, alleviation, reversal, prevention and/or delay of onset or recurrence of any symptom of a predetermined medical condition. In other words, “treating” a condition encompasses both therapeutic and prophylactic intervention against the condition, including facilitation of patient recovery from the condition.

The term “fecal microbiota transplantation (FMT)” or “stool transplant” refers to a medical procedure during which fecal matter containing live fecal microorganisms (bacteria, fungi, viruses, and the like) obtained from a healthy individual is transferred into the gastrointestinal tract of a recipient to restore healthy gut microflora that has been disrupted or destroyed by any one of a variety of medical conditions, for example, excess body weight or obesity and its related disorders. Typically, the fecal matter from a healthy donor is first processed into an appropriate form for the transplantation, which can be made through direct deposit into the lower gastrointestinal tract such as by colonoscopy, or by nasal intubation, or through oral ingestion of an encapsulated material containing processed (e.g., dried and frozen or lyophilized) fecal material.

The term “effective amount,” as used herein, refers to an amount of a substance that produces a desired effect (e.g., an inhibitory or suppressive effect on the growth or proliferation of one or more undesirable bacterial species for which the substance (e.g., an anti-bacterial agent) is used or administered. The effects include the prevention, inhibition, or delaying of any pertinent biological process during bacterial proliferation to any detectable extent. The exact amount will depend on the nature of the substance (the active agent), the manner of use/administration, and the purpose of the application, and will be ascertainable by one skilled in the art using known techniques as well as those described herein. In another context, when an “effective amount” of one or more beneficial or desirable bacterial species are artificially introduced into a composition intended to be introduced into the gastrointestinal tract of a patient, e.g., to be used in FMT, it is meant that the amount of the pertinent bacteria being introduced is sufficient to confer to the recipient health benefits such as reduced recovery time or reduced needs for therapeutic intervention for a pertinent disorder such as excessive body weight or obesity, including but not limited to medication (such as an appetite suppressant) and any of the variety of therapies such as behavior and communication therapy, educational therapy, family therapy, speech or physical therapy, and the like.

The term “inhibiting” or “inhibition,” as used herein, refers to any detectable negative effect on a target biological process, such as RNA/protein expression of a target gene, the biological activity of a target protein, cellular signal transduction, cell proliferation, and the like. Typically, an inhibition is reflected in a decrease of at least 10%, 20%, 30%, 40%, 50%, 60%, 70%, 80%, 90% or greater in the target process (e.g., growth or proliferation of a microorganism of certain species, for example, one or more of the bacterial species shown in Table 1), or any one of the downstream parameters mentioned above, when compared to a control. “Inhibition” further includes a 100% reduction, i.e., a complete elimination, prevention, or abolition of a target biological process or signal. The other relative terms such as “suppressing,” “suppression,” “reducing,” “reduction,” “decrease,” “decreasing,” “lower,” and “less” are used in a similar fashion in this disclosure to refer to decreases to different levels (e.g., at least 10%, 20%, 30%, 40%, 50%, 60%, 70%, 80%, 90% or greater decrease compared to a control level, i.e., the level before suppression) up to complete elimination of a target biological process or signal. On the other hand, terms such as “activate,” “activating,” “activation,” “increase,” “increasing,” “promote,” “promoting,” “enhance,” “enhancing,” “enhancement,” “higher,” and “more” are used in this disclosure to encompass positive changes at different levels (e.g., at least 10%, 20%, 30%, 40%, 50%, 60%, 70%, 80%, 90%, 100%, 200%, or greater such as 3, 5, 8, 10, 20-fold increase compared to a control level (before activation), for example, the control level of one or more of the bacterial species shown in Table 1) in a target process or signal. In contrast, the term “substantially the same” or “substantially lack of change” indicates little to no change in quantity from a comparison basis (such as a standard control value), typically within ±10% of the comparison basis, or within ±5%, 4%, 3%, 2%, 1%, or even less variation from the comparison basis.

The term “anti-bacterial agent” refers to any substance that is capable of inhibiting, suppressing, or preventing the growth or proliferation of bacterial species, e.g., any one shown in Tables 2, 4, and 5. Known agents with anti-bacterial activity include various antibiotics that generally suppress the proliferation of a broad spectrum of bacterial species as well as agents such as antisense oligonucleotides, small inhibitory RNAs, and the like that can inhibit the proliferation of specific bacterial species. The term “anti-bacterial agent” is similarly defined to encompass both agents with broad spectrum activity of killing virtually all species of bacteria and agents that specifically suppress proliferation of target bacteria species. Such specific anti-bacterial agent may be short polynucleotide in nature (e.g., a small inhibitory RNA, microRNA, miniRNA, lncRNA, or an antisense oligonucleotide) that is capable of disrupting the expression of a key gene in the life cycle of a target bacterial species and is therefore capable of specifically suppressing or eliminating the species only without substantially affecting other closely related bacterial species.

As used herein, the term “about” denotes a range of value that is +/−10% of a specified value. For instance, “about 10” denotes the value range of 9 to 11 (10+/−1).

### DETAILED DESCRIPTION OF THE INVENTION

### I. Introduction

The invention provides novel methods and compositions for assessing risk of obesity and type 2 diabetes (T2D) in individuals, especially those who have not been diagnosed with obesity or T2D, as well as for assessing individuals with obesity and T2D whether or not their condition is correlated with and potentially caused or exacerbated by a certain profile of bacteriome, or profile of pertinent bacterial species found in their gastrointestinal tract. Upon conclusion of such assessment, the individuals deemed to have an increased risk for obesity or T2D may receive treatment to prophylactically reduce or eliminate such risk and prevent or delay the onset of the conditions. Similarly, individuals already suffering from obesity and/or T2D upon being determined as having the condition or conditions in a microbiome-dependent nature may receive appropriate therapy to alleviate their symptoms in severity, extent, and/or duration. For example, the prophylactic or therapeutic treatment regimen may involve artificially modifying the level of pertinent bacterial species in a person's gastrointestinal tract, such as increasing the amount or level of “beneficial” bacteria or suppressing the amount or level of “detrimental” bacteria by way of fecal microbiota transplantation (FMT) treatment, in order to confer health benefits to the individuals being tested and treated.

### II. Treatment Methods by Modulating Bacterial Level

The discovery by the present inventors reveals the direct correlation between medical conditions such as obesity and T2D and the profile of certain bacterial species (e.g., those shown in Tables 1-5) in patients' gut. This revelation enables different methods for preventing and treating obesity and T2D as well as the pertinent symptoms, especially for aiding individuals with heightened risk for obesity and T2D or obese/T2D patients to benefit from different treatment regimens such as medication and/or various therapies, by adjusting or modulating the level of these bacterial species in the patient's GI tract via, e.g., an FMT procedure, to either deliver to the patients' gastrointestinal tract an effective amount of one or more of the “beneficial” or desirable bacterial species or to decrease the level of one or more “detrimental” or undesirable bacterial species by delivering an anti-bacterial agent to suppress the target bacterial species. In some cases, the composition used in FMT infusion is derived from a mixture of fecal materials from at least two donors having a desirable GI tract profile of beneficial bacterial species (e.g., from two lean donors) instead of from one single donor.

For example, one or more of desirable bacterial species such as some shown in Table 1 or 3 may be introduced from an exogenous source into a material being prepared for use in FMT so that the level of the bacterial species in the transfer material reaches a desired level (e.g., to reach at least about 0.01%, 0.02%, 0.05%, 0.10%, 0.20%, 0.40%, 0.50%, 0.60%. 0.80%, 1.0%, 2.0%, 3.0%, 4.0%, 5.0%, 6.0%, 7.0%, 8.0%, 8.5%, 9.0%, or 10% of total bacteria in the material) before it is processed for use in FMT for the prophylaxis or treatment of obesity and T2D for reducing obesity/T2D risk or alleviate obesity/T2D symptoms in an individual. In some cases, the beneficial bacterial species may be obtained from a bacterial culture in a sufficient quantity and then formulated into a suitable composition for delivery into a recipient's gut. Similar to FMT, such composition can be introduced into a patient by oral, nasal, or rectal administration.

On the other hand, certain bacterial species (e.g., some shown in Tables 2, 4, and 5) are found to rise in their relative abundance as a result of the presence of obesity/T2D or heightened risk of obesity/T2D. Thus, obesity/T2D patients or those at heightened risk for obesity/T2D are treated to reduce the level of these bacterial species in order to ameliorate the patients' symptoms related to the condition or to prevent/delay/reduce the likelihood of the onset of the condition. There are several options to reduce the level of these bacterial species: first, the patient may be given a specific anti-bacterial agent to specifically kill or suppress the targeted bacterial species, thereby lowering the level of these bacteria. Second, the patient may be first given an anti-bacterial agent, such as a broad spectrum antibiotic to kill or suppress all bacterial species, or a specific anti-bacterial agent to specifically kill or suppress the targeted bacterial species; then a composition may be administered to the patient (e.g., by FMT) to introduce a well-balanced mixed bacterial culture into the GI tract of the patient.

Each of these options can be performed in one combined step to achieve the first and second treatment method goals, i.e., to increase the level of certain bacterial species and to decrease the level of certain other bacterial species, using one single composition (such as processed fecal material from an FMT donor) containing the pertinent bacterial species within the appropriate ratio range to one another.

Immediately upon completion of the step of introducing an effective amount of the desired bacterial species into a patient's GI tract (e.g., via an FMT procedure) and/or the step of suppressing undesirable bacterial level, the recipient may be further monitored by continuous testing of the level or relative abundance of the bacterial species in the stool samples on a daily or weekly or monthly basis for up to 6 months post-procedure while the clinical symptoms of obesity/T2D being treated as well as the general health status of the patient are also being monitored in order to assess treatment outcome and the corresponding levels of relevant bacteria in the recipient's GI tract: the level of bacterial species (one or more of those shown in Tables 1-5) may be monitored in connection with observation of health benefits achieved such as improvement in body weight, blood pressure, blood glucose, lipid, and cholesterol levels.

### III. Assessing Obesity/T2D Risk and Microbiome-Dependence

The present inventors discovered that the altered profile of certain bacterial species in a person's GI tract can indicate the presence or risk of obesity/T2D, even though the person may not have been diagnosed with obesity or T2D: the level or relative abundance of certain bacterial species (such as one or more of the species shown in Table 1) have been revealed to indicate a subject's heightened risk for later developing obesity/T2D or a subject's obesity/T2D is related to the gut profile of bacterial species (i.e., “microbiome-dependent”) when properly calculated using certain specified mathematic tools, e.g., as described herein.

Once the obesity/T2D risk assessment is made, for example, an individual is deemed to have microbiome-dependent obesity/T2D or is at an increased risk of later developing obesity/T2D, appropriate treatment steps can be taken as a measure to address the disease or the heightened risk for the individual. For example, the individual may be given medication such as blood sugar reducing drugs, insulin sensitizing drugs, and/or appetite suppressant drugs or may be given compositions that comprise an effective amount of (1) one or more of the beneficial bacterial species or (2) an antibacterial substance that suppresses the detrimental bacterial species, either by FMT or by an alternative administration method, such that the bacterial profile in the patient's GI tract will be modified to one that is favorable for the outcome of bodyweight reduction and prevention of T2D or alleviation of T2D symptoms.

### IV. Kits and Compositions

The present invention provides kits and compositions useful for reducing the risk of obesity and type 2 diabetes (T2D) or for treating obesity and T2D in a subject. The kit comprises two or more containers, each containing a different composition comprising an effective amount of a different the bacterial species or a different combination of bacterial species selected from the group consisting of Faecalibacterium prausnitzi, Bifidobacterium longum, Eubacterium halli, Bifidobacterium bifidum, Roseburia intestinalis, Eubacterium eligens, Lachnospiraceae bacterium_5_1_63FAA, Eubacterium ventriosum, and Roseburia hominis. The compositions are formulated for being introduced into the gastrointestinal tract of a recipient, e.g., by oral administration or by direct delivery using a suppository. In addition to the bacterial species named above, the compositions may further include one or more therapeutic agents effective for reducing blood glucose, sensitizing insulin response, and suppressing appetite to further facilitate managing the risk of T2D and obesity.

The present invention also provides novel kits and compositions that can be used for assessing a patient's likelihood of later develop obesity and T2D or for assessing whether a patient's obesity/T2D is microbiome-dependent. Typically, the kit comprises reagents for detecting one or more of the bacterial species set forth in Tables 1-5. For example, a kit is provided that comprises (1) a first container containing a first composition comprising a first reagent for detection of one of the bacterial species set forth in Tables 1-5, and (2) a second container containing a second composition comprising a second and different reagent for detection of one of the bacterial species set forth in Tables 1-5. Optionally, a third reagent for detecting the bacterial species of Tables 1-5 may be included in the kit. When the kit is intended for detecting two or more bacterial species of Tables 1-5, additional compositions comprising additional reagents may be included in the kit so as to allow the user to detect and measure the presence and level of multiple bacterial species. In some variations, the first and second (and optionally more) reagents may be included in one single composition.

In some cases, the reagents comprise a set of oligonucleotide primers for amplification of a polynucleotide sequence from any one of the bacterial species set forth in Tables 1-5. For example, the reagents may be primers and/or probes for a polymerase chain reaction (PCR) as the amplification reaction, such as quantitative PCR. Typically, such reagents may comprise a set of oligonucleotide primers for PCR of a polynucleotide sequence derived from, and preferably unique to, each one of the pertinent bacterial species (such as any one or more of bacterial species selected from Tables 1-5).

As an alternative, the means for detecting one or more of the bacterial species set forth in Tables 1-5 is metagenomics sequencing, and the kit includes compositions comprising one or more reagents suitable for metagenomics sequencing for the pre-elected bacterial species (one or more listed in Tables 1-5). For example, a kit may contain detection reagents for analyzing the bacterial species comprising: (i) Clostridium bartlettii, Haemophilus parainfluenzae, Escherichia coli, Lachnospiraceae bacterium 5_1_63FAA, Eubacterium ventriosum or, (ii) Clostridium bartlettii or, (iii) Haemophilus parainfluenzae or, (iv) Escherichia coli or, (v) Lachnospiraceae bacterium 5_1_63FAA or, (vi) Eubacterium ventriosum or, (vii) Clostridium bartlettii, Haemophilus parainfluenzae, Escherichia coli, Lachnospiraceae bacterium 5_1_63FAA or, (viii) Clostridium bartlettii, Haemophilus parainfluenzae, Escherichia coli, Eubacterium ventriosum or, (ix) Clostridium bartlettii, Haemophilus parainfluenzae, Lachnospiraceae bacterium 5_1_63FAA, Eubacterium ventriosum or, (x) Clostridium bartlettii, Escherichia coli, Lachnospiraceae bacterium 5_1_63FAA, Eubacterium ventriosum or, (xi) Haemophilus parainfluenzae, Escherichia coli, Lachnospiraceae bacterium 5_1_63FAA, Eubacterium ventriosum or, (xii) Clostridium bartlettii, Lachnospiraceae bacterium 5_1_63FAA, Eubacterium ventriosum or, (xiii) Clostridium bartlettii, Haemophilus parainfluenzae, Lachnospiraceae bacterium 5_1_63FAA or, (xiv) Haemophilus parainfluenzae, Escherichia coli, Lachnospiraceae bacterium 5_1_63FAA or, (xv) Clostridium bartlettii, Haemophilus parainfluenzae, Escherichia coli or, (xvi) Haemophilus parainfluenzae, Escherichia coli.

### Examples

The following examples are provided by way of illustration only and not by way of limitation. Those of skill in the art will readily recognize a variety of non-critical parameters that could be changed or modified to yield essentially the same or similar results.

### BACKGROUND

The purpose of this study is to determine how human gut bacteriome is associated with obesity and type 2 diabetes (T2D). The practical use of the invention includes assessing disease risks associated with obesity and T2D based on the presence and quantity of certain bacterial species in the test subject's gastrointestinal tract and assessing whether in a test subject obesity and T2D is associated with gut microbiome, especially bacteriome.

### Example 1: Machine Learning Model to Predict Risk of Obesity with Type 2 Diabetes Mellitus

### Methods

### Cohort Description and Study Subjects

A total of 123 Chinese adults were recruited, including 68 subjects having both obesity and type 2 diabetes mellitus (ObT2) subjects (BMI >28 kg/m²) and 55 healthy lean subjects (lean control, BMI <23 kg/m²). The study was approved by The Joint Chinese University of Hong Kong, New Territories East Cluster Clinical Research Ethics Committee (The Joint CUHK-NTEC CREC, CREC Ref. No: 2016.607). All subjects consented to donate fecal samples and to the questionnaire investigation, where written informed consents were obtained. Fecal samples from the study subjects were stored at −80° C. for downstream microbiome analyses.

### Fecal DNA Extraction and DNA Sequencing

Fecal bacterial DNA was extracted by Maxwell® RSC PureFood GMO and Authentication Kit (Promega) with modifications to increase the yield of DNA.

Approximately 100 mg from each stool sample was pretreated: stool sample suspended in 1 ml ddH₂O and pelleted by centrifugation at 13,000×g for 1 min. Washed sample was added 800 μl TE buffer (PH 7.5), 16 μl beta-Mercaptoethanol and 250 U lyticase sufficiently mixed and digestion at 37° C. for 90 minutes. Pelleted by centrifugation at 13,000×g for 3 minutes.

After pretreatment, precipitate was re-suspended in 800 μl CTAB buffer (Maxwell® RSC PureFood GMO and Authentication Kit following manufacturer's instructions) and mixed well. After samples were heated at 95° C. for 5 minutes and cooled down, nucleic acid was released from the samples by vortexing with 0.5 mm and 0.1 mm beads at 2850 rpm for 15 minutes. Following this, 40 μl Proteinase K and 20 μl RNase A were added and nucleic acid digested at 70° C. for 10 minutes. Finally, supernatant was obtained after centrifugation at 13,000×g, 5 minutes and placed in a Maxwell® RSC instrument for DNA extraction. The extracted fecal DNA was used for ultra-deep metagenomics sequencing via Ilumina Novaseq 6000 (Novogene, Beijing, China).

### Quality Control of Raw Sequences

Raw sequence reads were trimmed by Trimmomatic¹ (v0.38) firstly and then separation of non-human reads from contaminant host reads. There were some steps to acquire clean reads: 1) Remove adapters; 2) Scan the read with a 4-base wide sliding window, removing reads when the average quality per base drop below 20; 3) Drop reads below the 50 bases long. Trimmed sequence reads were mapped to human genome (Reference database: GRCh38 p12) by KneadData (v0.7.2) to remove reads originated from the host. Pair-end two reads were concatenated together.

### Analysis of the Bacterial Microbiome

Profiling of the composition of bacterial communities was performed on metagenomic trimmed reads via MetaPhlAn2 (v2.7.5)². Mapping reads to clade-specific markers gene and annotation of species pangenomes was done through Bowtie2 (v2.3.4.3)³. The output table contained bacterial species and its relative abundance in different levels, from kingdom to species level. The resulting data were analyzed in R v3.6.1 using tidyverse (v1.2.1)⁴, ggpubr (v0.2, website: github.com/kassambara/ggpubr) and phyloseq (v1.24.2)⁵. Differential bacterial species were compared between ObT2 subjects with lean controls via Linear discriminant analysis effect size (LEfSe) analysis⁶. Another method of bacterial taxonomy annotation was used as an alternative analysis of the bacterial microbiome. In this method, Kraken2 (v2.0.8-beta) was used to generate a species-level community composition. The reference bacterial genome was downloaded from NCBI RefSeq on Nov. 5, 2019, and the database was built with default parameters. Each query was thereafter classified to a taxon with the highest total hits of k-mer matched by pruning the general taxonomic trees affiliated with mapped genomes. Multivariate association with linear models (MaAsLin2) was used to identify associations between clinical metadata and microbial abundance while controlling for confounders.

### Machine Learning Model

Random forest (RF) was chosen to build an assessment model using fecal microbes because of its superior performance for classification with binary features. Random Forest⁷ is one of the most popular approaches in metagenomic data analysis to identify the discriminative features and build prediction models. As a widely used ensemble learning algorithm, Random Forest consists of a series of classification and regression trees (CARTs) to form a strong classifier. A subset of data randomly sampled from the original dataset with replacement is known as bootstrap sampling, applying to build the trees. When the training dataset for the current tree is drawn by the bootstrap method,

$$1 - {( 1 - \frac{1}{N} )}^{N}$$

observations are left out from the overall dataset. With infinite N, there are 36.8% data not occurred in the training samples called out-of-bag (OOB) observations, which would not be used for constructing the trees. In addition, extra randomness introduced to the random forest as each decision tree splits nodes based on a random subset of features selected from the overall features. The features with the least Gini (Gini are used to evaluate the purity of the node) would be utilized to split the nodes in each iteration to generate the trees. With different subsets of data and features, the algorithm is able to train different trees and obtain the final classification by averaging the result from the tree models. In addition to the prediction model, Random Forest has the capability to assess the importance of variables⁸. The OOB observations are used to estimate the classification error for each tree in the forest. To measure the importance of a given variable, the values of the variable in the OOB data are randomly altered, and then the changed OOB data is used to generate new predictions. The difference of the error rate between the altered and the original OOB observations divided by the standard error is calculated as the importance of a variable. To classify a new sample, the features of the sample passed down to each tree to estimate the probability for classification. The Random Forest used the average probability of all trees to determine the final result of the classification.

The importance value of each species to the classification model was evaluated by recursive feature elimination. According to descending importance value, the selected species were added one by one to the random forest model if its Pearson correlation value with any already existing probe in the model was <0.7. Each time a new feature was added to the model, the performance of the model was re-evaluated using 10-fold cross-validation. These models were compared in terms of binary classifiers with Area Under the Curve (AUC) in Receiver Operating Characteristic (ROC) curves. The final model was chosen when best accuracy and kappa were achieved. These analysis was done using R packages randomForest v4.6-14⁷ and pROC v1.15.3⁹.

### Results

### Gut Bacterial Profile is Different Between Lean Subjects and ObT2 Subjects

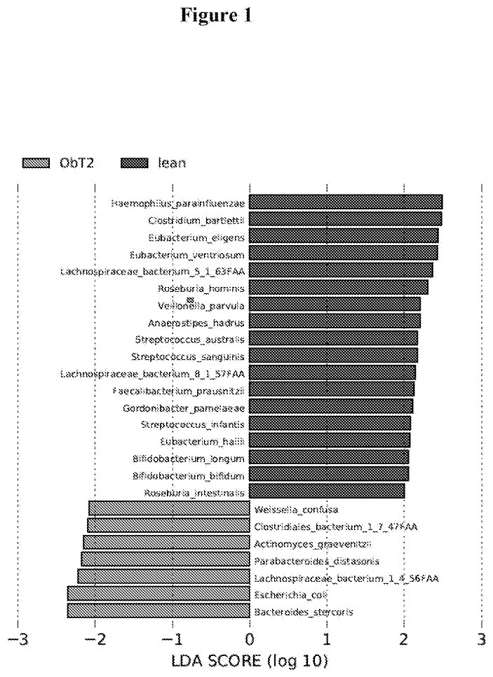

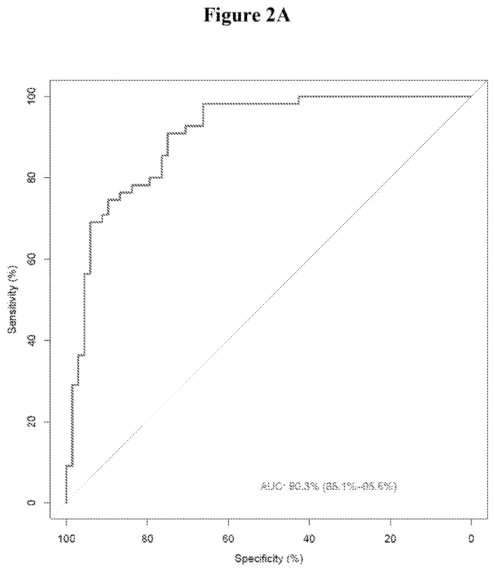

With the MetaPhlAn2 and LEfSe analysis, it was discovered that bacterial species Faecalibacterium prausnitzii, Bifidobacterium longum, Eubacterium hallii, Bifidobacterium bifidum, Roseburia intestinalis, Eubacterium eligens, Lachnospiraceae bacterium 5_1_63FAA, Eubacterium ventriosum, Roseburia hominis, Clostridium bartlettii, Anaerostipes hadrus, Gordonibacter pamelaeae, Veillonella parvula, Haemophilus parainfluenzae, Lachnospiraceae bacterium 8_1_57FAA, Streptococcus sanguinis, Streptococcus australis and Streptococcus infantis (FIG. 1, Table 1) showed higher relative abundance in lean controls compared with ObT2 subjects. In contrast, the species Escherichia coli, Parabacteroides distasonis, Bacteroides stercoris, Lachnospiraceae bacterium 1_4_56FAA, Clostridiales bacterium 1_7_47FAA, Weissella confusa and Actinomyces graevenitzii (FIG. 1, Table 2) were enriched in ObT2 subjects compared with lean controls.

TABLE 1
Bacterial species enriched in lean controls compared
with ObT2 subjects (via MetaPhlAn2 method)

| | | Mean Relative | Mean Relative |
|---|---|---|---|
| | | Abundance in Lean | Abundance in |
| Bacterial Species | NCBI: txid | Subjects (%) | ObT2 subjects (%) |
| Faecalibacterium prausnitzii | 853 | 6.020 | 3.664 |
| Bifidobacterium longum | 216816 | 5.499 | 2.705 |
| Eubacterium hallii | 39488 | 2.302 | 1.653 |
| Bifidobacterium bifidum | 1681 | 2.075 | 1.191 |
| Roseburia intestinalis | 166486 | 1.252 | 0.383 |
| Eubacterium eligens | 39485 | 0.899 | 0.274 |
| Lachnospiraceae bacterium 5_1_63FAA | 658089 | 0.808 | 0.316 |
| Eubacterium ventriosum | 39496 | 0.527 | 0.266 |
| Roseburia hominis | 301301 | 0.362 | 0.124 |
| Clostridium bartlettii | 261299 | 0.151 | 0.019 |
| Anaerostipes hadrus | 649756 | 0.147 | 0.123 |
| Gordonibacter pamelaeae | 471189 | 0.091 | 0.066 |
| Veillonella parvula | 29466 | 0.076 | 0.067 |
| Haemophilus parainfluenzae | 729 | 0.063 | 0.007 |
| Lachnospiraceae bacterium 8_1_57FAA | 665951 | 0.059 | 0.020 |
| Streptococcus sanguinis | 1305 | 0.053 | 0.035 |
| Streptococcus australis | 113107 | 0.045 | 0.022 |
| Streptococcus infantis | 68892 | 0.018 | 0.016 |

Arranged by mean relative abundance in healthy lean subjects

TABLE 2
Bacterial species enriched in ObT2 subjects compared
with lean controls (via MetaPhlAn2 method)

| | | Mean Relative | Mean Relative |
|---|---|---|---|
| | | Abundance in Lean | Abundance in |
| Bacterial Species | NCBI: txid | Subjects (%) | ObT2 subjects (%) |
| Escherichia coli | 562 | 0.683 | 3.875 |
| Parabacteroides distasonis | 823 | 0.177 | 0.476 |
| Bacteroides stercoris | 46506 | 0.152 | 0.258 |
| Lachnospiraceae bacterium 1_4_56FAA | 658655 | 0.029 | 0.162 |
| Clostridiales bacterium 1_7_47FAA | 457421 | 0.016 | 0.020 |
| Weissella confuse | 1583 | 0.009 | 0.063 |
| Actinomyces graevenitzii | 55565 | 0.008 | 0.020 |

Arranged by mean relative abundance in healthy lean subjects

With an alternative method of using the Kraken2 to annotate the bacteriome taxonomy, it was discovered that a series of species showed higher relative abundance in lean controls compared with ObT2 subjects (Table 3), while some species showed higher relative abundance in ObT2 subjects compared with lean controls (Table 4).

TABLE 3
Bacterial species enriched in lean controls compared with ObT2 subjects (via Kraken2 method)

| | | Mean Relative | Mean Relative |
|---|---|---|---|
| | | Abundance in | Abundance in |
| Bacterial Species | NCBI: txid | Lean Subjects (%) | ObT2 subjects (%) |
| Faecalibacterium prausnitzii | 853 | 8.193 | 6.192 |
| Anaerostipes hadrus | 649756 | 6.559 | 2.967 |
| Roseburia intestinalis | 166486 | 2.524 | 1.177 |
| Anaerostipes Lachnospiraceae bacterium GAM79 | | 1.642 | 1.066 |
| Longibaculum sp. KGMB06250 | 2584943 | 1.584 | 1.195 |
| Anaerostipes Lachnospiraceae bacterium Choco86 | | 1.407 | 1.193 |
| Eubacterium eligens | 39485 | 1.283 | 0.556 |
| Roseburia hominis | 301301 | 1.147 | 0.672 |
| Ruminococcus bicirculans | 1160721 | 1.061 | 0.429 |
| Longibaculum Erysipelotrichaceae bacterium | | 0.436 | 0.320 |
| GAM147 | | | |
| Dysosmobacter welbionis | 2093857 | 0.372 | 0.313 |
| Mordavella sp. Marseille-P3756 | 2086584 | 0.364 | 0.255 |
| Anaerostipes Lachnospiraceae bacterium KM106-2 | | 0.238 | 0.188 |
| Bifidobacterium breve | 1685 | 0.196 | 0.085 |
| Dialister sp. 5BBH33 | 2582419 | 0.185 | 0.074 |
| Dialister sp. Marseille-P5638 | 2161821 | 0.164 | 0.067 |

Arranged by mean relative abundance in healthy lean subjects

TABLE 4
Bacterial species enriched in ObT2 subjects compared
with lean controls (via Kraken2 method)

| | | Mean Relative | Mean Relative |
|---|---|---|---|
| | | Abundance in | Abundance in |
| Bacterial Species | NCBI: txid | Lean Subjects (%) | ObT2 subjects (%) |
| Escherichia coli | 562 | 0.300 | 1.826 |
| Parabacteroides distasonis | 823 | 0.206 | 0.829 |
| Bacteroides caccae | 47678 | 0.120 | 0.469 |
| Acidaminococcus intestini | 187327 | 0.086 | 0.844 |

Arranged by mean relative abundance in healthy lean subjects

Bacteria listed in Table 1, 2, 3, and 4 can be used in different combinations to determine the risk of obesity and T2D. For example, the relative abundance can be determined using as a panel of qPCR primer or by metagenomics sequencing to calculate the risk.

Furthermore, bacteria listed in Table 1 and 3 can be administered to subjects with obesity and T2D or at risk for developing obesity and T2D to ameliorate symptoms of obesity and T2D or reduce risk for later developing obesity and T2D. Conversely, bacteria listed in Table 2 and 4 can be targeted for suppression in subjects with obesity and T2D or at risk for developing obesity and T2D to ameliorate symptoms of obesity and T2D or reduce risk for later developing obesity and T2D.

### Machine Learning Model for Prediction of ObT2

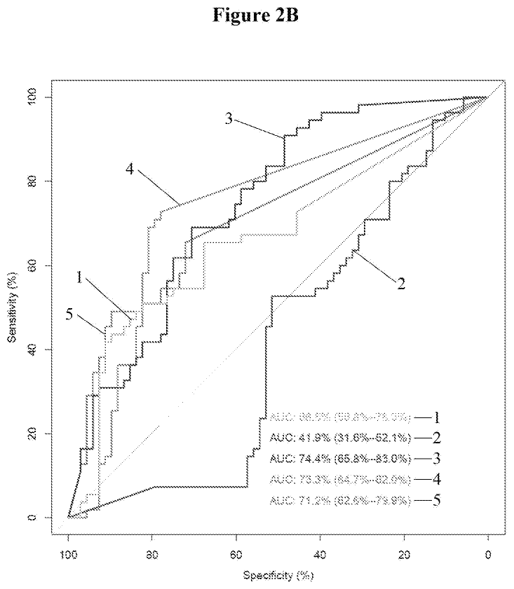

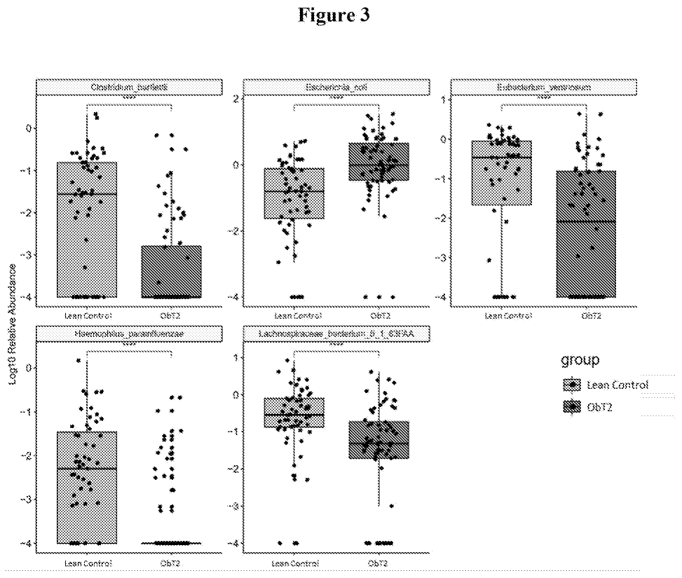

Five bacterial markers were used in the machine learning model, including Clostridium bartlettii, Haemophilus parainfluenzae, Escherichia coli, Lachnospiraceae bacterium_5_1_63FAA and Eubacterium ventriosum (Table 5). The final model has an Area Under the Curve (AUC) in Receiver Operating Characteristic (ROC) curves of 90.3% (FIG. 2A). The relative abundance of these bacteria in ObT2 and lean controls are shown in FIG. 3.

TABLE 5
Bacterial species included in the machine
learning model for assessment of ObT2

| Bacterial Species | NCBI: txid |
|---|---|
| Clostridium bartlettii | 261299 |
| Haemophilus parainfluenzae | 729 |
| Escherichia coli | 562 |
| Lachnospiraceae bacterium_5_1_63FAA | 658089 |
| Eubacterium ventriosum | 39496 |

TABLE 6
Relative abundance of species listed in Table 5 in ObT2 subjects
(n = 68) and healthy controls (n = 55)

| | Clostridium | Haemophilus | Escherichia | Lachnospiraceae | Eubacterium |
|---|---|---|---|---|---|
| group | bartlettii | parainfluenzae | coli | bacterium 5_1_63FAA | ventriosum |
| healthy | 0.0296 | 0.27867 | 0.02165 | 0.01204 | 0.7527 |
| healthy | 0.25876 | 0.01642 | 0 | 0 | 0 |
| healthy | 0.19599 | 0.00072 | 1.43966 | 2.52485 | 0.35478 |
| healthy | 0 | 0.11703 | 0.11244 | 0.67608 | 1.98294 |
| healthy | 0.33898 | 0 | 0.26297 | 0.946 | 0 |
| healthy | 0.12933 | 0.12172 | 0.00165 | 0.23919 | 0.92215 |
| healthy | 0.11755 | 0 | 0 | 0.09795 | 0.37323 |
| healthy | 0.19106 | 0.25634 | 0.79825 | 0.1079 | 0.23754 |
| healthy | 0 | 0 | 0.01505 | 0.43822 | 2.28673 |
| healthy | 0 | 0 | 1.16157 | 0.05555 | 0.69697 |
| healthy | 1.75862 | 0.04586 | 0.00102 | 0.04975 | 0.33822 |
| healthy | 0.05204 | 0.01773 | 0.00846 | 0.26247 | 0.34563 |
| healthy | 0.06973 | 0.00492 | 5.2128 | 0.79431 | 1.25227 |
| healthy | 0.09485 | 0.08673 | 0 | 4.13323 | 0.38806 |
| healthy | 0 | 0.07612 | 4.87662 | 1.28431 | 0 |
| healthy | 0.03441 | 0.00552 | 0.62247 | 4.55139 | 0.09116 |
| healthy | 0.01785 | 0.05967 | 0.07854 | 1.36365 | 1.68023 |
| healthy | 0.48675 | 0.00808 | 0.03868 | 0.46776 | 1.07835 |
| healthy | 4.00E−04 | 0 | 0.2096 | 1.83935 | 0.07044 |
| healthy | 2.1737 | 0.00069 | 3.60153 | 0.42189 | 0.95281 |
| healthy | 0.00216 | 0.00158 | 0.14493 | 0.97061 | 0.17637 |
| healthy | 0.11732 | 0 | 0.01786 | 0.15672 | 1.13513 |
| healthy | 0.25782 | 0.06897 | 1.38229 | 0 | 0 |
| healthy | 0.2587 | 0.00222 | 1.45494 | 0.71959 | 0.00075 |
| healthy | 0 | 0.02923 | 0.01457 | 0.7039 | 0.78025 |
| healthy | 0.33034 | 0.2872 | 0.19885 | 0.40285 | 1.26742 |
| healthy | 0 | 0 | 0.74226 | 0.19035 | 0.9715 |
| healthy | 0 | 0.02793 | 0.67496 | 0.20797 | 0 |
| healthy | 0.0248 | 0 | 0.01021 | 1.53324 | 0 |
| healthy | 0 | 0.00705 | 0.16 | 0.51851 | 0.75731 |
| healthy | 0 | 0.00352 | 0.55808 | 0.48828 | 0.41098 |
| healthy | 0 | 0.02793 | 0.67496 | 0.20797 | 0 |
| healthy | 0.02696 | 0.0096 | 0.1979 | 0.14938 | 1.95176 |
| healthy | 0 | 0 | 0.24649 | 0.00643 | 1.08786 |
| healthy | 0 | 0 | 0.00298 | 0.19873 | 0.00798 |
| healthy | 0.01218 | 0.00275 | 0 | 1.09334 | 0.1322 |
| healthy | 0 | 0 | 0.04287 | 0.18373 | 0.05262 |
| healthy | 0.02914 | 0 | 0.12181 | 0.34414 | 0.03057 |
| healthy | 0 | 0.04012 | 0.08575 | 8.29347 | 0 |
| healthy | 0.0332 | 0 | 0.03607 | 0.13095 | 0.25454 |
| healthy | 0.25656 | 0.00359 | 0.94203 | 0.06663 | 0.70325 |
| healthy | 0.01831 | 0.00906 | 0.37177 | 0.28265 | 0.39699 |
| healthy | 0.19756 | 0.00113 | 1.4421 | 0.28992 | 0.19207 |
| healthy | 0 | 0.00307 | 0.03764 | 0.13363 | 1.10985 |
| healthy | 0.00858 | 7.00E−04 | 1.79135 | 0.13717 | 0.01535 |
| healthy | 0.01016 | 0 | 0.02659 | 2.06365 | 0.75151 |
| healthy | 0.10325 | 0 | 1.13853 | 0.02087 | 0.87985 |
| healthy | 0.15411 | 0.00166 | 0.15564 | 0.11519 | 0.09432 |
| healthy | 0 | 0.0071 | 3.79215 | 2.6036 | 0 |
| healthy | 0.2216 | 0.00062 | 1.96975 | 0.00499 | 0 |
| healthy | 0.00741 | 1.47578 | 0.08166 | 0.11382 | 0 |
| healthy | 0 | 0.00607 | 0.15808 | 0.50387 | 0.77305 |
| healthy | 0.15461 | 0.29756 | 0.00444 | 0.81753 | 0.18109 |
| healthy | 0.0276 | 0.04914 | 0.0529 | 0.14323 | 0.91406 |
| healthy | 0.11582 | 0.00661 | 0.38519 | 0.3509 | 0.16907 |
| ObT2 | 0 | 0.01075 | 0.5345 | 0 | 0 |
| ObT2 | 0.04258 | 0 | 6.15803 | 0 | 0 |
| ObT2 | 0 | 0 | 2.45261 | 0.01927 | 0 |
| ObT2 | 0.00252 | 0 | 2.47106 | 0.04428 | 0.07455 |
| ObT2 | 0 | 0 | 0.65573 | 0.07068 | 0.00518 |
| ObT2 | 0 | 0 | 29.96304 | 0.05026 | 0 |
| ObT2 | 0 | 0 | 13.15843 | 0.02448 | 0.12404 |
| ObT2 | 0 | 0 | 0.87054 | 0.38395 | 0 |
| ObT2 | 0 | 0.03585 | 2.79786 | 0.42931 | 0 |
| ObT2 | 0.00143 | 0 | 18.05658 | 0.33301 | 0 |
| ObT2 | 0 | 0 | 8.42611 | 0 | 0 |
| ObT2 | 0 | 0 | 0.67825 | 0.76145 | 0 |
| ObT2 | 0 | 0.0276 | 0.16978 | 1.10605 | 0.01268 |
| ObT2 | 0 | 0 | 1.24562 | 0 | 0 |
| ObT2 | 0 | 0 | 0.31194 | 0 | 0 |
| ObT2 | 0 | 0 | 1.62331 | 0.65451 | 0 |
| ObT2 | 0 | 0 | 0.18205 | 0.12997 | 0 |
| ObT2 | 0.3151 | 0 | 0.75719 | 0.04259 | 0.22795 |
| ObT2 | 0 | 0 | 0.02718 | 0.0883 | 0.14336 |
| ObT2 | 0 | 0 | 0 | 0.03284 | 0.61414 |
| ObT2 | 0.07498 | 0 | 1.37707 | 0.02611 | 0.0206 |
| ObT2 | 0 | 0.00045 | 24.62422 | 0.04696 | 1.30001 |
| ObT2 | 0 | 0.01487 | 0.29944 | 0.0401 | 0.18085 |
| ObT2 | 0.00708 | 0 | 6.43953 | 0.03086 | 0.33507 |
| ObT2 | 0.00367 | 0 | 0.21828 | 0.03048 | 0.15441 |
| ObT2 | 0.0018 | 0 | 0.11912 | 0.0265 | 0.15986 |
| ObT2 | 0.00978 | 0 | 0.22194 | 0.03158 | 0.77763 |
| ObT2 | 0 | 0 | 5.98915 | 0.14729 | 0 |
| ObT2 | 0 | 0 | 2.59521 | 0.6936 | 1.00047 |
| ObT2 | 0 | 0 | 0.16005 | 0.10346 | 0.01652 |
| ObT2 | 0 | 0 | 3.6718 | 0 | 0 |
| ObT2 | 0 | 0 | 0 | 2.59271 | 0 |
| ObT2 | 0 | 0 | 0.08445 | 2.43355 | 0 |
| ObT2 | 0.00075 | 0.00058 | 0.527 | 0.10704 | 0.07124 |
| ObT2 | 0 | 0.02253 | 2.37697 | 0 | 0 |
| ObT2 | 0.02827 | 0 | 0.96544 | 0 | 0 |
| ObT2 | 0 | 0 | 6.05192 | 0.04955 | 0.14894 |
| ObT2 | 0 | 0.00152 | 0.73099 | 0.10935 | 0.42012 |
| ObT2 | 0.01457 | 0 | 0.87633 | 0.06552 | 4.35595 |
| ObT2 | 0 | 0 | 7.98931 | 0.05723 | 0.02143 |
| ObT2 | 0 | 0.01132 | 1.36138 | 0.03063 | 0.59437 |
| ObT2 | 0 | 0 | 12.45388 | 0.04837 | 0.6283 |
| ObT2 | 0 | 0 | 0.71767 | 0.01032 | 0.04012 |
| ObT2 | 0 | 0.003 | 0.54456 | 0.01774 | 0.02802 |
| ObT2 | 0 | 0 | 0.65966 | 0 | 0 |
| ObT2 | 0.00868 | 0.00327 | 0.35088 | 0.02117 | 0 |
| ObT2 | 0.01809 | 0.00829 | 1.27878 | 0.44054 | 4.29162 |
| ObT2 | 0 | 0 | 6.64529 | 0.18318 | 0.04079 |
| ObT2 | 0 | 0 | 0.32862 | 0 | 0 |
| ObT2 | 0 | 0 | 1.00237 | 9.00E−04 | 0.05682 |
| ObT2 | 0 | 0 | 7.22579 | 0 | 0 |
| ObT2 | 0 | 0 | 0.11234 | 4.10017 | 0.00166 |
| ObT2 | 0 | 0 | 1.70946 | 0.7073 | 0 |
| ObT2 | 0.00721 | 0 | 6.06937 | 0 | 0 |
| ObT2 | 0 | 0.0048 | 3.27664 | 0.20826 | 0.228 |
| ObT2 | 0.00012 | 0 | 5.90566 | 0.02048 | 0.00098 |
| ObT2 | 0.00866 | 0 | 0.3329 | 2.08675 | 0 |
| ObT2 | 0.67533 | 0 | 0.6182 | 1.25412 | 0.02045 |
| ObT2 | 0 | 0 | 0.04445 | 0.15072 | 0 |
| ObT2 | 0 | 0 | 0.27749 | 0.52829 | 0 |
| ObT2 | 0 | 0.21155 | 10.75605 | 0.16604 | 0 |
| ObT2 | 0 | 0 | 0.80499 | 0.05328 | 0 |
| ObT2 | 0 | 0 | 3.18223 | 0.04096 | 1.0141 |
| ObT2 | 0.08693 | 0.10494 | 0.50201 | 0.15503 | 0.5183 |
| ObT2 | 0 | 0 | 2.66649 | 0.48606 | 0.0414 |
| ObT2 | 0 | 0 | 4.0522 | 0 | 0 |
| ObT2 | 0.01243 | 0 | 0.41955 | 0 | 0.39028 |
| ObT2 | 0 | 0 | 34.34773 | 0.02903 | 0.05471 |

TABLE 7
Relative abundance of species listed in Table 2 in new subjects

| | Clostridium | Haemophilus | Escherichia | Lachnospiraceae | Eubacterium |
|---|---|---|---|---|---|
| | bartlettii | parainfluenzae | coli | bacterium 5_1_63FAA | ventriosum |
| Example 1: | 0 | 0.00052 | 0.38401 | 0 | 0 |
| FB002 | | | | | |
| Example 2: | 0.5499 | 0.19779 | 0.20368 | 0.00858 | 0.00263 |
| H45 | | | | | |

This machine learning model can be used to (1) predict the risk of being ObT2 in a subject who is not obese or do not have type 2 diabetes mellitus (T2DM) at the time of test and (2) assess whether ObT2 of a subject is microbiome-dependent in a subject who is already obese or have T2DM.

The following steps will be carried out:
1. Obtain a set of training data by determine the relative abundance of species selected from Table 4 in a cohort of obese with type 2 diabetes (ObT2) subjects and lean controls. Species selected from Table 5 should comprise of: (i) Clostridium bartlettii, Haemophilus parainfluenzae, Escherichia coli, Lachnospiraceae bacterium 5_1_63FAA, Eubacterium ventriosum (all 5 species; AUC: 90.3%; FIG. 2A) or, (ii) Clostridium bartlettii (AUC: 71.2%; FIG. 2B (red)) or, (iii) Haemophilus parainfluenzae (AUC: 73.3%; FIG. 2B (light blue)) or, (iv) Escherichia coli (AUC: 74.4%; FIG. 2B (green)) or, (v) Lachnospiraceae bacterium 5_1_63FAA (AUC: 41.9%; FIG. 2B (dark blue)) or, (vi) Eubacterium ventriosum (AUC: 66.5.2%; FIG. 2B (orange)) or, (vii) Clostridium bartlettii, Haemophilus parainfluenzae, Escherichia coli, Lachnospiraceae bacterium 5_1_63FAA (AUC: 87.7%) or, (viii) Clostridium bartlettii, Haemophilus parainfluenzae, Escherichia coli, Eubacterium ventriosum (AUC: 86.9%) or, (ix) Clostridium bartlettii, Haemophilus parainfluenzae, Lachnospiraceae bacterium 5_1_63FAA, Eubacterium ventriosum (AUC: 86.2%) or, (x) Clostridium bartlettii, Escherichia coli, Lachnospiraceae bacterium 5_1_63FAA, Eubacterium ventriosum (AUC: 88.8%) or, (xi) Haemophilus parainfluenzae, Escherichia coli, Lachnospiraceae bacterium 5_1_63FAA, Eubacterium ventriosum (AUC: 85.0%) or, (xii) Clostridium bartlettii, Lachnospiraceae bacterium 5_1_63FAA, Eubacterium ventriosum (AUC: 86.7%) or, (xiii) Clostridium bartlettii, Haemophilus parainfluenzae, Lachnospiraceae bacterium 5_1_63FAA (AUC: 85.0%) or, (xiv) Haemophilus parainfluenzae, Escherichia coli, Lachnospiraceae bacterium 5_1_63FAA (AUC: 84.1%) or, (xv) Clostridium bartlettii, Haemophilus parainfluenzae, Escherichia coli (AUC: 86.4%) or, (xvi) Haemophilus parainfluenzae, Escherichia coli (AUC: 85.6%).
2. To determine the risk of ObT2 in a subject who is not obese or do not have T2DM, or to determine whether the already existing ObT2 of a subject is microbiome-related, determine the relative abundance of these species.
3. Compare the relative abundance of these species in the subject with the training data using random forest model.
4. Decision trees will be generated by random forest from the training data. The relative abundances will be run down the decision trees and generate a risk score. If more than 50% trees in the model consider the subject resemble the ObT2 group, the outcome will be “the subject is deemed to have an increased risk of ObT2” or “the existing ObT2 of the subject is microbiome-dependent”. If less than 50% trees in the model consider the subject resemble the ObT2 group, the outcome will be “the subject is deemed to have a low risk of ObT2” or “the existing ObT2 of the subject is unlikely to be microbiome-dependent”.

### Study 1:

The relative abundance of 5 species listed in Table 5 from ObT2 subjects (n=68) and healthy controls (n=55) was determined by metagenomics sequencing and taxonomy assigned as described in methods (relative abundance listed in Table 6). Decision trees were generated by random forest from data in Table 6 with parameter: trees=801, mtry=3.

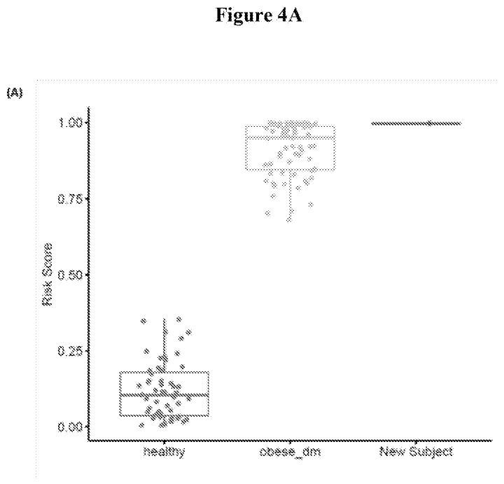

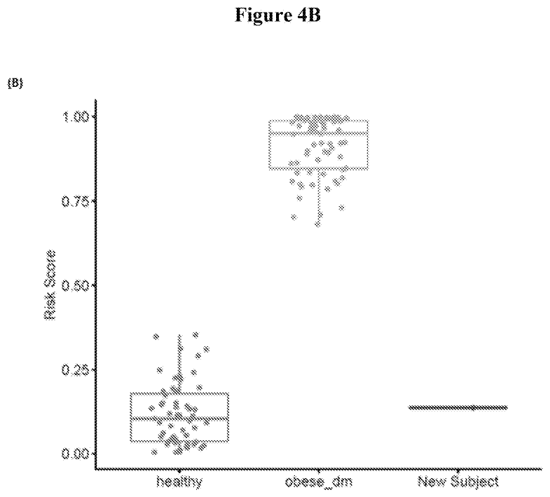

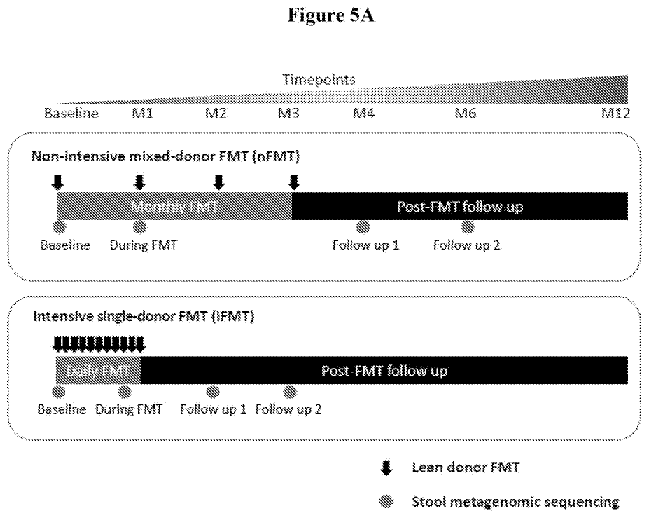

The risk of having ObT2 in a 50-year-old male subject (FB002) was determined. The relative abundance of the 5 species listed in Table 5 in fecal sample of this subject was determined by metagenomics sequencing and taxonomy assigned as described in method. Relative abundance of the 5 species in this subject is shown in Table 7. The relative abundances were run down the decision trees and a risk score was generated using the relative abundances in Table 6 as training data. The score of the subject was 0.997 (FIG. 4A), and therefore the subject was deemed to be likely to be ObT2. This subject has a BMI of 41.7, and was diagnosed with T2DM.

### Study 2:

The relative abundance of 5 species listed in Table 5 from ObT2 subjects (n=68) and healthy controls (n=55) was determined by metagenomics sequencing and taxonomy assigned as described in methods (relative abundance listed in Table 6). Decision trees were generated by random forest from data in Table 6 with parameter: trees=801, mtry=3.

The risk of having ObT2 in a 45-year-old male subject (H45) was determined. The relative abundance of the 5 species listed in Table 5 in fecal sample of this subject was determined by metagenomics sequencing and taxonomy assigned as described in method. Relative abundance of the 5 species in this subject is shown in Table 7. The relative abundances were run down the decision trees and a risk score was generated using the relative abundances in Table 6 as training data. The score of the subject was 0.137 (FIG. 4B), and therefore the subject was deemed to have a low risk of ObT2. This subject has a BMI of 21.09 and no T2DM.

### Example 2: Mixed-Donor FMT Induced Increase in Abundance and Diversity of Butyrate-Producing Bacteria

### Background

The purpose of this study is to determine how human gut bacteriome is associated with obesity and type 2 diabetes (T2D). The practical use of the invention includes assessing disease risks associated with obesity and T2D based on the presence and quantity of certain bacterial species in the test subject's gastrointestinal tract and assessing whether in a test subject obesity and T2D is associated with gut microbiome, especially bacteriome.

### Methods

### Study Subjects and Study Design

Two FMT studies, randomized controlled trials of non-intensive FMT (nFMT) and intensive FMT study (iFMT), were conducted and compared. In both studies, obese subjects aged 18-70 years with a body mass index of >28 kg/m² and <45 kg/m² were recruited from a tertiary referral centre in Hong Kong (ClinicalTrials.gov NCT03789461, NCT03127696). Patients with the use of weight loss medication in the preceding one year, and those with immunodeficiency syndrome, contraindications to oesophago-gastro-duodenoscopy (OGD), history of food allergies, severe organ failure including decompensated cirrhosis, inflammatory bowel disease, kidney failure, epilepsy, known malignancy in recent 2 years and active sepsis were excluded. Subjects taking antibiotics or probiotics within 12 weeks of screening, or those on sodium-glucose co-transporter-2 inhibitors, Glucagon-like peptide-1 (GLP-1) receptor agonists or proton-pump inhibitors at randomisation were also excluded. Antibiotics, probiotics or prebiotics were prohibited during study period. During the study, patients remained on the same dose of oral hypoglycemic drugs and lipid lowering drugs. All patients gave written informed consent. The Joint Chinese University of Hong Kong New Territories East Cluster Clinical Research Ethics Committee approved this study (2016.136-T and 2018.444).

### FMT Regimen

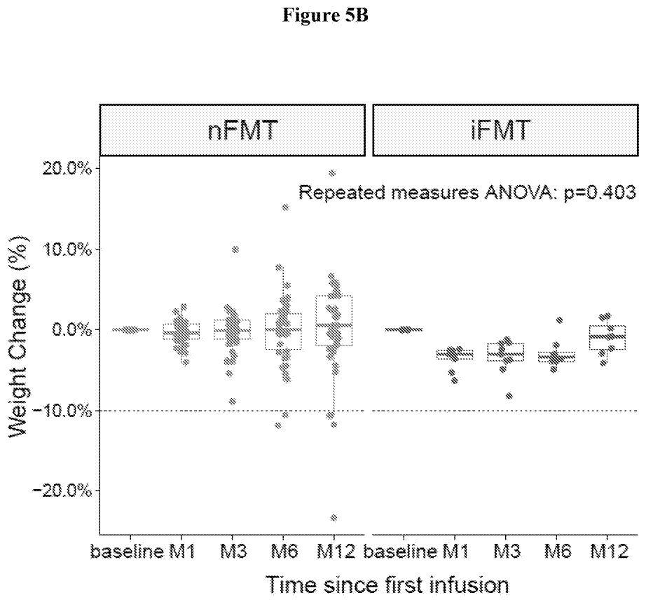

In the nFMT study, recipients received non-intensive FMT infusions once per month for 4 months (total 4 FMTs). FMT infusions were derived from a mixture of at least two lean donors. In the iFMT study, each subject received a 3-day antibiotics preparation (Vancomycin, Metronidazole and Amoxicilin, 500 mg each, 3 times daily), followed by 5-day consecutive single-donor FMT weekly for four weeks (total 20 FMTs). Serial stool samples were collected from all recipients at four time points (baseline, one month after first FMT, one month after last FMT, and 2-3 months after last FMT) (FIG. 5A).

### FMT Donors

FMT solution was derived from lean donors with a BMI <23 kg/m² according to a set of stringent criteria as previously described¹⁰. Stool samples of eligible donors were collected within 4 hours of bowel movements, which were inspected visually for suitability (formed stool, no blood or mucous). Donor stool was homogenised with isotonic saline and glycerol, filtered, and then stored at −80° C. FMT solutions from single-donor or pooling of donors' stool (50 gm stool in 100-200 ml saline) was infused into the distal duodenum via Oesophago-gastro-duodenoscopy (OGD) under conscious sedation.

### Shotgun Metagenomics Sequencing and Fecal Microbiota Profiling

Faecal DNA for bacterial metagenomics sequencing was isolated using Maxwell® RSC PureFood GMO and Authentication Kit according to manufacturer's instructions. DNA libraries were constructed through the processes of end repairing, purification, and PCR amplification and sequenced by Illumina Novaseq 6000 with paired-end 150 bp sequencing strategy by Novogene, Beijing, China, generating 93.5±15.2 million (mean±standard deviation, SD) raw reads per sample. Metagenomics reads were quality-filtered and trimmed using Trimmomatic¹¹ (v0.38) and decontaminated against human genome (Reference: hg38) by Kneaddata (v0.7.2, website: bitbucket.org/biobakery/kneaddata/wiki/Home). MetaPhlAn2¹² (v2.6.0) was used to generate a species-level metagenomic analysis. StrainPhlAn¹³ (v3) was used to generate a strain-level metagenomic analysis. The resulting abundance tables were processed in R v3.6.0 and tidyverse¹⁴ (v1.2.1), ggpubr¹⁵ (v0.2) and phyloseq¹⁶ (v1.24.2) R packages. Raw sequencing data were accessible at NCBI under Bio project PRJNA644456 and RJNA633456.

### Covariation of Bacteria Species

Covariation of bacteria species after FMT was identified as previously described.¹⁷ Briefly, the relative change of bacteria species was computed as the difference in relative abundance between each post FMT sample and the baseline sample. The correlation of relative change of bacteria species at each post FMT timepoint were then tabulated. Covariation was assumed when correlation was significant (p<0.05 Pearson's correlation) at both one month after last FMT and 2-3 months after last FMT.

### Statistical Analysis

Continuous variables were expressed in mean±SD or median (25th to 75th percentile, P25-P75) as appropriate, whereas categorical variables were presented as number (percentage). Repeated measures ANOVA (with logarithmic transformation for skewed variables) was applied for between-group comparisons. Wilcoxon rank sum test was used to investigate significant differences between groups comparisons. Wilcoxon signed-rank test was used to compare data between different time points within the same treatment. Linear discriminant analysis Effect Size¹⁸ (LEfSe) was used to determine the taxa between two groups. Differences between pre-FMT and post-FMT samples were assessed by bray curtis distance after centred log-ratio transformation¹⁹. All statistical tests were two-sided. Statistical significance was taken as P<0.05.

### Result

### Study Subjects

In the nFMT study, a total of 38 obese subjects with BMI ranging from 28.0 to 44.9 kg/m² received fecal infusions from 2-5 lean donors. In the iFMT study, nine obese subjects with a BMI ranging from 31.9 to 41.5 kg/m² received fecal infusions from one single lean donor. Recipient baseline characteristics are summarized in Table 8.

TABLE 8
Subject baseline characteristics in each study

| | nFMT | | iFMT | |
|---|---|---|---|---|
| Study | (n = 38) | | (n = 9) | |
| Male, n (%) | 27 | (71.1) | 14 | (70.0) |
| Age, years (P25-P75) | 55 | (44.2-61.8) | 46 | (37-53) |
| Smoker/Ex-smoker, n (%) | 5 (13.2)/8 (21.1) | | 6 | (30.0) |
| Alcoholic drinker/ | 2 (5.3)/3 (7.9) | | 1 | (5.0) |
| Ex-drinker, n (%) | | | | |
| BMI, kg/m² (P25-P75) | 32 | (29.3-35.7) | (31.9-41.5, range) | |
| Fasting glucose, | 6.4 | (5.5-7) | 5.8 | (5.3-6.3) |
| mmol/l (P25-P75) | | | | |
| HbA1C, % (P25-P75) | 7.2 | (6.5-8.6) | 6.6 | (5.9-6.9) |
| Cholesterol | | | | |
| Total, mmol/l (P25-P75) | 4.4 | (3.7-5.0) | 4.7 | (4.3-5.6) |
| LDL, mmol/l (P25-P75) | 2.2 | (1.8-2.6) | 2.4 | (2.2-3.3) |
| HDL, mmol/l (P25-P75) | 1.4 | (1.1-1.5) | 1.3 | (1.1-1.6) |
| Triglycerides, mmol/l (P25-P75) | 1.6 | (1.1-2.0) | 2.0 | (1.5-2.7) |
| ALT, IU/I (P25-P75) | 34 | (20.5-42.5) | 33 | (24-57) |

Data expressed as number of subjects (%) or median (P25-P75).
Abbreviation: BMI, body mass index; LDL, low-density lipoprotein; HDL, high-density lipoprotein; ALT, alanine aminotransferase.

### Impact of Intensive Versus Non-Intensive FMT on Weight Loss in Obese Subjects

After FMT intervention, obese recipients in both studies showed heterogenous weight loss compared to baseline (nFMT 3.1%±4.8% vs iFMT 4.8%±1.7%, mean±sd, maximum weight loss during 52 weeks of follow-up). No significant improvement of weight loss was observed in subjects receiving iFMT compared with nFMT (Repeated measures ANOVA, p=0.403, FIG. 5B). None of the nine subjects receiving iFMT achieved ≥10% weight loss whereas 13.2% (5 out of 38) subjects receiving nFMT achieved ≥10% weight loss (maximum weight loss during 52 weeks of follow-up, FIG. 5B).

### Intensive FMT LED to Increased Number of Lean Donor-Derived Species and Resemblance to Donor Microbiome Profile

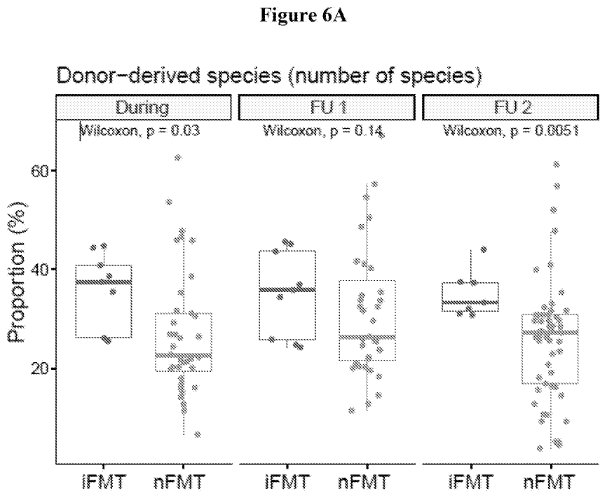

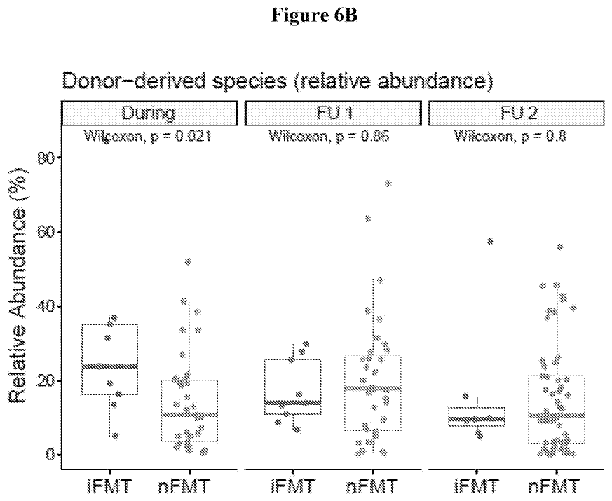

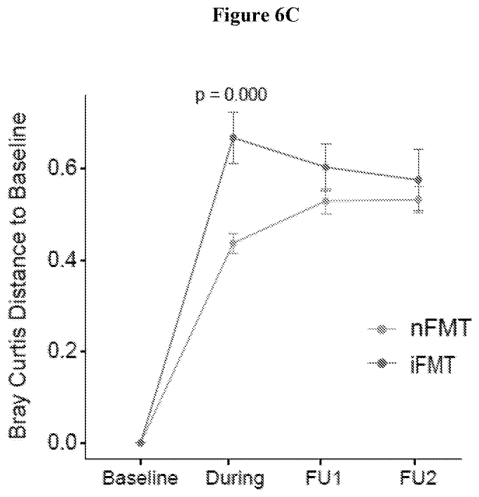

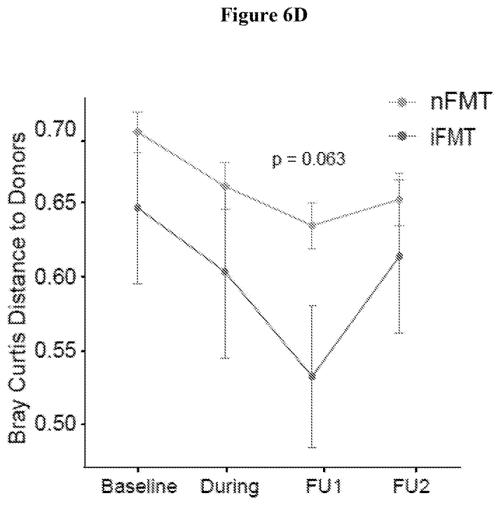

Obese subjects receiving iFMT harbored significantly more donor-derived bacteria species at one month after first FMT and 2-3 months after the last FMT infusion, compared with obese subjects receiving nFMT (p=0.03 and p<0.01, Wilcoxon rank sum test, FIG. 6A). The aggregated abundance of donor-derived bacteria species at one month after first FMT was significantly higher in subjects receiving iFMT than nFMT (p=0.02, Wilcoxon rank sum test, FIG. 6B). The bray-curtis distance between baseline and post FMT samples in subjects receiving iFMT was significantly larger than that in subjects receiving nFMT, suggesting that iFMT conferred more changes in overall bacterial composition (p<0.001, Wilcoxon rank sum test, FIG. 6C). At one month after last FMT, the bray-curtis distance between iFMT recipients' post-FMT samples and the samples of respective donors was significantly smaller than nFMT recipients (p=0.06, Wilcoxon rank sum test, FIG. 6D), suggesting that the bacteriome profile after iFMT showed more resemblance to that of their respective donors, compared with the bacteriome profile after nFMT.

Mixed-Donor FMT was Associated with Increased Abundance and Diversity of Butyrate-Producing Bacteria in Obese Subjects

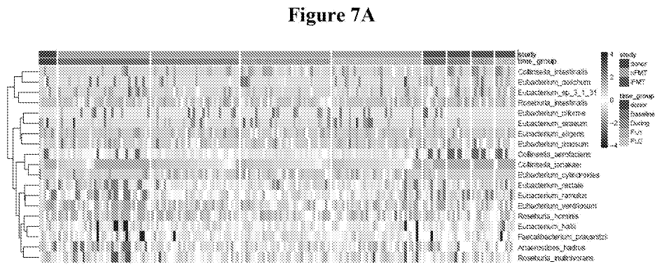

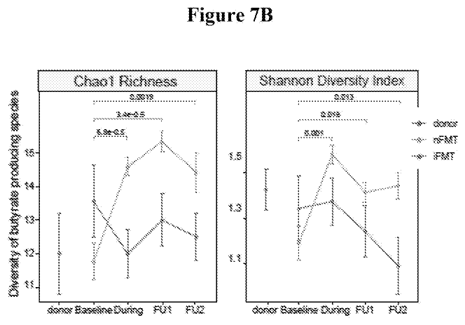

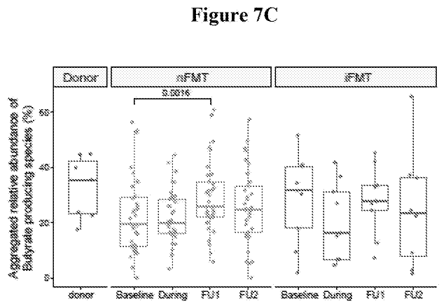

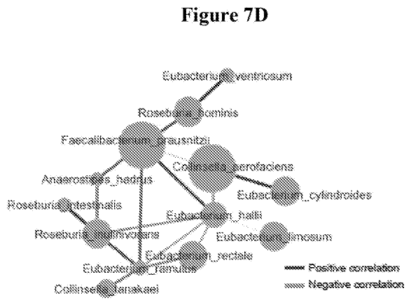

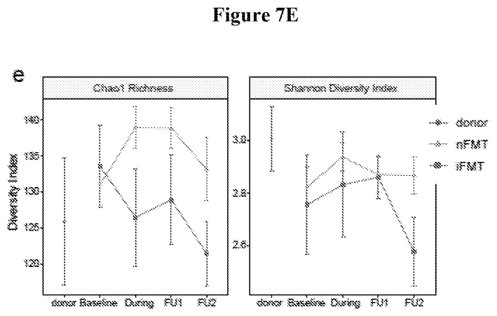

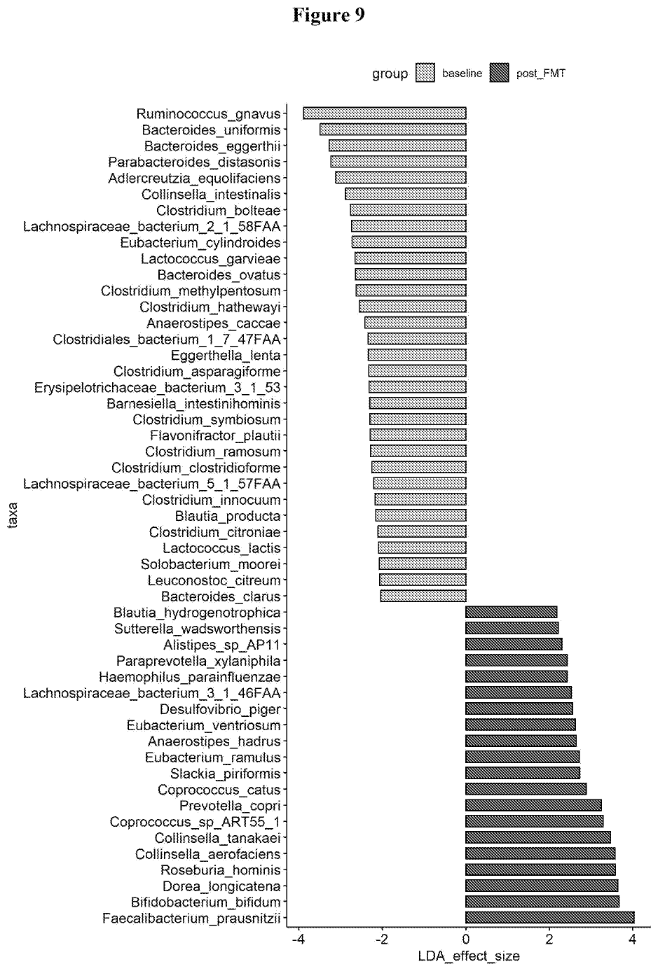

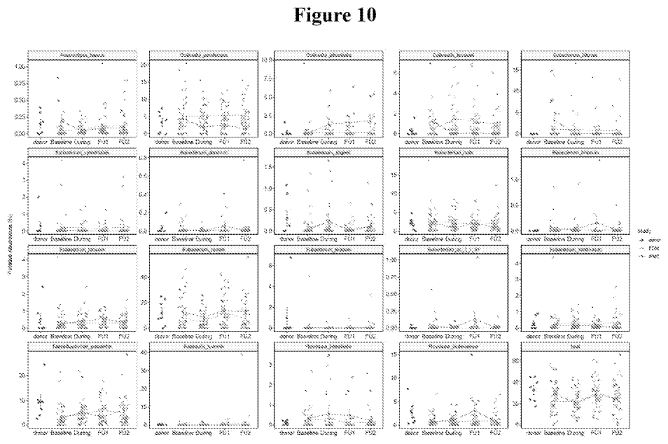

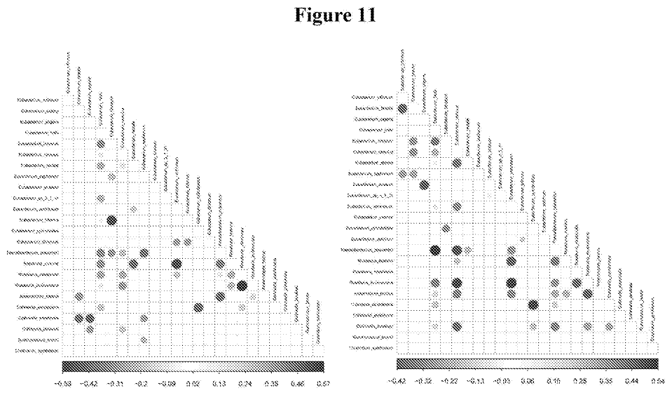

In the nFMT study whereby each subject received fecal infusions from 2-5 lean donors, a substantial increase of butyrate-producing bacteria including Eubacterium species, Roseburia hominis, Anaerostipes hadrus²⁰, Faecalibacterium prausnitzii²¹ and Collinsella species²² was observed (FIG. 7A, FIG. 9, LDA>2, p<0.05). The Chao 1 richness and Shannon diversity, as well as the aggregated abundance of butyrate-producing species were significantly higher in post-FMT samples, compared to baseline samples (p<0.01 and p<0.05, Wilcoxon signed-rank test, FIG. 7B, C). In contrast, in the iFMT study whereby recipients received single-donor FMT, there was no significant increase in Chao 1 richness, Shannon diversity or aggregated abundance of butyrate-producing species (FIG. 7A-C, FIG. 10). Abundance of Bifidobacterium bifidum, which has been shown to interact with butyrate-producing bacteria by cross-feeding interactions²³, significantly increased in samples following nFMT, but not iFMT (FIG. 9, LDA>2, p<0.05). The change of major butyrate-producing species consistently correlated at one month and 2-3 months after last FMT (FIG. 7D, FIG. 11), indicating that these species maintained covariation despite massive disturbance following FMT. The richness of overall bacteriome also significantly increased in nFMT recipients at one month after first FMT and 2-3 months after the last FMT (p<0.01 and p<0.05, Wilcoxon signed-rank test, FIG. 7E)¹⁰ while no significant alterations was observed iFMT recipients. These results indicate that mixed-donor FMT, but not single-donor FMT, was associated with increased abundance and diversity of butyrate-producing bacteria in obese subjects.

### Intensive FMT LED to Increased Replacement of Butyrate-Producing Bacteria Strains Compared to Non-Intensive FMT

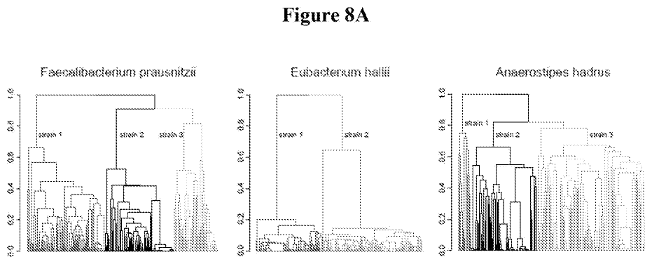

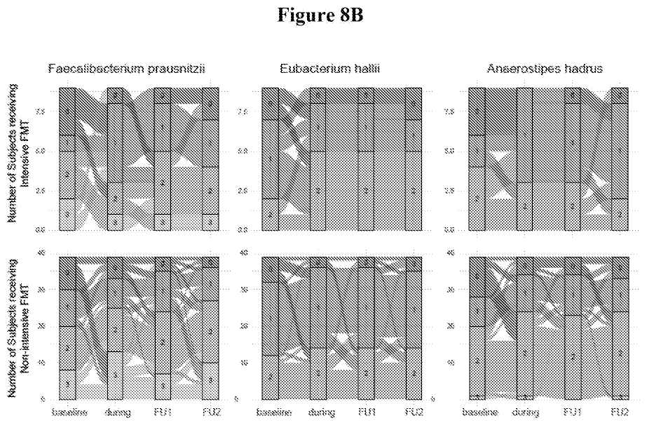

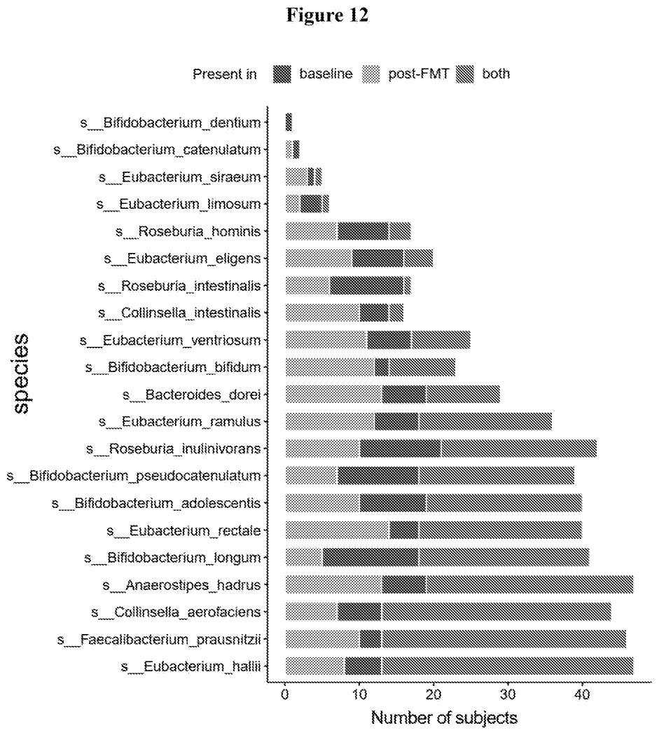

The inventors then looked for strain engraftment or replacement in recipients of major butyrate-producing bacteria. Eubacterium hallii, Faecalibacterium prausnitzii, and Anaerostipes hadrus were present in over 50% of FMT recipients and formed distinct clusters based on SNP haplotype profile (FIG. 8A, FIG. 12). Subjects receiving intensive FMT had higher rate of strain engraftment or replacement compared with subjects receiving non-intensive FMT at second follow-up (E. hallii: 77.8% vs 26.3%, F. prausnitzii: 66.7% vs 57.9, Anaerostipes hadrus: 88.9% vs 52.6%, intensive vs non-intensive FMT, FIG. 8B). This indicate that intensive FMT was more effective in replacing original strain with donor-derived strains.

### Discussion

This study aimed to explore whether intensive FMT could improve donor engraftment and induce weight loss, and to evaluate factors affecting FMT outcome in subjects with obesity. It was discovered that intensive FMT did not induce more weight loss compared with mixed-donor monthly FMT. Despite intensive FMT induced significant higher numbers of lean donor-derived species, the aggregated abundance of donor-derived species was only transiently increased compared to mixed-donor monthly FMT. On the contrary, mixed-donor monthly FMT was more effective in inducing increase of butyrate-producing bacteria and induced significant weight loss (≥10%) in a subset of obese recipients. Among all baseline factors, recipient's baseline microbiome composition showed the strongest power in predicting weight change. High baseline B. dorei was associated with more weight loss after FMT.

Mixed-donor intensive FMT was shown to increase FMT efficacy in diseases such as ulcerative colitis²⁴. The change of microbiome profiles was compared in patients with obesity following single-donor intensive FMT or mixed-donor monthly FMT. The hypothesis was that antibiotic preparation followed by a frequent, intensive course of FMT may enhance microbiota alterations and improve clinical outcomes in subjects with obesity.

As expected, intensive FMT led to increased number of donor-derived species, compared with mixed-donor FMT. However, the aggregated abundance of donor-derived species only showed significant difference one month since first FMT, but not during follow-up visits compared with mixed-donor FMT. Similarly, the overall compositional changes and the resemblance to donor microbiome profiles to in subjects receiving intensive FMT peaked during FMT and one month since last FMT, but reduced to similar levels as mixed-donor FMT two to three months since last FMT infusion. These data indicate that single-donor intensive FMT only led to transient improvement of microbiome profiles compared with mixed-donor monthly FMT.

Butyrate producing bacteria are a group of commensal bacteria with the ability of converting nondigestible carbohydrates to butyrate²⁵, the latter was shown to reduce the level of circulating cholesterol^{26, 27}. Extensive increase of multiple butyrate producing species was observed, as indicated by both increased abundance and increased diversity, after mixed-donor monthly FMT. In subjects receiving intensive single-donor FMT, the increase of butyrate-producing species showed high inter-person variabilities. This was similar with previous single-donor FMT studies, where increase was only observed for a few butyrate-producing species²⁸⁻³⁰. A previous study reported associations were observed for licorisoflavan A, pyrrole, and p-cresol sulfate, and the levels of these metabolites were significantly associated with F. prausnitzii strain shift³¹. However, no significant association of butyrate-producing strains and clinical outcomes was observed, which may relate to factors such as limited sample size, or strain variation insufficient to affect clinical manifestations. By deciphering the co-variation patterns in recipient fecal microbiota, the inventors showed that several butyrate-producing species acted as a co-varying unit that retained positive correlation with each other following FMT. Despite the same selection criteria, the microbiome profile of the lean donors varied largely in terms of the presence and abundance of butyrate-producing species. Co-infusion of butyrate-producing species by pooling stool samples from multiple donors may therefore increase the colonization of butyrate-producing species in the recipients' gut.

All patents, patent applications, and other publications, including GenBank Accession Numbers and the like, cited in this application are incorporated by reference in the entirety for all purposes.

### LIST OF REFERENCES

1. Bolger A M, Lohse M, Usadel B. Trimmomatic: a flexible trimmer for Illumina sequence data. Bioinformatics 2014; 30:2114-20.
2. Truong D T, Franzosa E A, Tickle T L, et al. MetaPhlAn2 for enhanced metagenomic taxonomic profiling. Nat Methods 2015; 12:902-3.
3. Langmead B, Salzberg S L. Fast gapped-read alignment with Bowtie 2. Nat Methods 2012; 9:357-9.
4. Hadley W, Mara A, Jennifer B, et al. Welcome to the Tidyverse. Journal of Open Source Software 2019; 4:1686.
5. McMurdie P J, Holmes S. phyloseq: an R package for reproducible interactive analysis and graphics of microbiome census data. PLOS One 2013; 8: e61217.
6. Segata N, Izard J, Waldron L, et al. Metagenomic biomarker discovery and explanation. Genome Biol 2011; 12: R60.
7. Breiman L. Random Forests. Machine Learning 2001; 45:5-32.
8. Cutler D R, Edwards Jr T C, Beard K H, et al. Random forests for classification in ecology. Ecology 2007; 88:2783-2792.
9. Robin X, Turck N, Hainard A, et al. pROC: an open-source package for R and S+ to analyze and compare ROC curves. BMC Bioinformatics 2011; 12:77.
10. Ng S C, Xu Z, Mak J W Y, et al. Microbiota engraftment after faecal microbiota transplantation in obese subjects with type 2 diabetes: a 24-week, double-blind, randomised controlled trial. Gut 2021.
11. Bolger A M, Lohse M, Usadel B. Trimmomatic: a flexible trimmer for Illumina sequence data. Bioinformatics 2014; 30:2114-20.
12. Truong D T, Franzosa E A, Tickle T L, et al. MetaPhlAn2 for enhanced metagenomic taxonomic profiling. Nat Methods 2015; 12:902-3.
13. Beghini F, McIver L J, Blanco-Miguez A, et al. Integrating taxonomic, functional, and strain-level profiling of diverse microbial communities with bioBakery 3. Elife 2021; 10.
14. Wickham H A M, Bryan J, et al. “Welcome to the tidyverse.”. ournal of Open Source Software 2019; 4 (43), 1686.
15. Wickham H. ggpubr: ‘ggplot2’ Based Publication Ready Plots.
16. McMurdie P J, Holmes S. Phyloseq: a bioconductor package for handling and analysis of high-throughput phylogenetic sequence data. Pac Symp Biocomput 2012:235-46.
17. Raman A S, Gehrig J L, Venkatesh S, et al. A sparse covarying unit that describes healthy and impaired human gut microbiota development. Science 2019; 365: eaau4735.
18. Segata N, Izard J, Waldron L, et al. Metagenomic biomarker discovery and explanation. Genome Biol 2011; 12: R60.
19. Gloor G B, Macklaim J M, Pawlowsky-Glahn V, et al. Microbiome Datasets Are Compositional: And This Is Not Optional. Front Microbiol 2017; 8:2224.
20. Kant R, Rasinkangas P, Satokari R, et al. Genome Sequence of the Butyrate-Producing Anaerobic Bacterium Anaerostipes hadrus PEL 85. Microbiology Resource Announcements 2015; 3.
21. Vital M, Karch A, Pieper D H. Colonic Butyrate-Producing Communities in Humans: an Overview Using Omics Data. mSystems 2017; 2: e00130-17, /msystems/2/6/msys.00130-17.atom.
22. Qin P P, Zou Y Q, Dai Y, et al. Characterization a Novel Butyric Acid-Producing Bacterium Collinsella aerofaciens Subsp. Shenzhenensis Subsp. Nov. Microorganisms 2019; 7.
23. Rivière A, Selak M, Lantin D, et al. Bifidobacteria and Butyrate-Producing Colon Bacteria: Importance and Strategies for Their Stimulation in the Human Gut. Frontiers in Microbiology 2016; 7.
24. Paramsothy S, Kamm M A, Kaakoush N O, et al. Multidonor intensive faecal microbiota transplantation for active ulcerative colitis: a randomised placebo-controlled trial. The Lancet 2017; 389:1218-1228.
25. Fu X, Liu Z, Zhu C, et al. Nondigestible carbohydrates, butyrate, and butyrate-producing bacteria. Crit Rev Food Sci Nutr 2019; 59: S130-S152.
26. Canfora E E, Jocken J W, Blaak E E. Short-chain fatty acids in control of body weight and insulin sensitivity. Nat Rev Endocrinol 2015; 11:577-91.
27. Kenny D J, Plichta D R, Shungin D, et al. Cholesterol Metabolism by Uncultured Human Gut Bacteria Influences Host Cholesterol Level. Cell Host Microbe 2020; 28:245-257 e6.
28. Kootte R S, Levin E, Salojärvi J, et al. Improvement of Insulin Sensitivity after Lean Donor Feces in Metabolic Syndrome Is Driven by Baseline Intestinal Microbiota Composition. Cell Metabolism 2017; 26:611-619.e6.
29. Dao M C, Everard A, Aron-Wisnewsky J, et al. Akkermansia muciniphila and improved metabolic health during a dietary intervention in obesity: relationship with gut microbiome richness and ecology. Gut 2016; 65:426-436.
30. Forslund K, Hildebrand F, Nielsen T, et al. Disentangling type 2 diabetes and metformin treatment signatures in the human gut microbiota. Nature 2015; 528:262-+.
31. Chen L, Wang D, Garmaeva S, et al. The long-term genetic stability and individual specificity of the human gut microbiome. Cell 2021; 184:2302-2315 e12.

1. A method for reducing the risk of obesity and type 2 diabetes (T2D) or treating obesity and T2D in a subject, comprising introducing into the subject's gastrointestinal tract an effective amount of one or more of the bacterial species selected from the group consisting of Faecalibacterium prausnitzi, Bifidobacterium longum, Eubacterium halli, Bifidobacterium bifidum, Roseburia intestinalis, Eubacterium eligens, Lachnospiraceae bacterium_5_1_63FAA, Eubacterium ventriosum, and Roseburia hominis.

2. The method of claim 1, wherein the introducing step comprises oral administration to the subject a composition comprising an effective amount of the one or more of the bacterial species.

3. The method of claim 1, wherein the introducing step comprises delivery to the small intestine, ileum, or large intestine of the subject a composition comprising an effective amount of the one or more of the bacterial species.

4. The method of claim 1, wherein the introducing step comprises fecal microbiota transplantation (FMT).

5. The method of claim 4, wherein the FMT comprises administration to the subject a composition comprising processed donor fecal material.

6. The method of claim 5, wherein the processed donor fecal material is from at least two lean donors.

7-11. (canceled)

12. A method for assessing risk of obesity and type 2 diabetes (T2D) in a subject, comprising:
(1) determining, in a stool sample from the subject, the level or relative abundance of one or more of the bacterial species set forth in Tables 1-5;
(2) determining the level or relative abundance of the same bacterial species in a stool sample from a reference cohort comprising subjects with obesity and T2D and subject without obesity and T2D;
(3) generating decision trees by random forest model using data obtained from step (2) and running the level or relative abundance of one or more of the bacterial species from step (1) down the decision trees to generate a score; and
(4) determining the subject with a score greater than 0.5 as having an increased risk for obesity and T2D, and determining the subject with a score no greater than 0.5 as having no increased risk for obesity and T2D.

13. The method of claim 12, wherein the one or more bacterial species comprise any two or three bacterial species set forth in Tables 1-5.

14. The method of claim 12, wherein the subject has not been diagnosed with obesity.

15. The method of claim 12, wherein the subject has not been diagnosed with T2D.

16. The method of claim 12, wherein each of steps (1) and (2) comprises metagenomics sequencing.

17. The method of claim 12, wherein each of steps (1) and (2) comprises polymerase chain reaction (PCR).

18-19. (canceled)

20. A method for assessing whether a subject has microbiome-dependent obesity and T2D, comprising:
(1) determining, in a stool sample from the subject, the level or relative abundance of one or more of the bacterial species set forth in Tables 1-5;
(2) determining the level or relative abundance of the same bacterial species in a stool sample from a reference cohort comprising subjects with obesity and T2D and subject without obesity and T2D;
(3) generating decision trees by random forest model using data obtained from step (2) and running the level or relative abundance of one or more of the bacterial species from step (1) down the decision trees to generate a score; and
(4) determining the subject with a score greater than 0.5 as having microbiome-dependent obesity and T2D, and determining the subject with a score no greater than 0.5 as having microbiome-independent obesity and T2D.

21. The method of claim 20, wherein the subject has been diagnosed with obesity.

22. The method of claim 20, wherein the subject has been diagnosed with T2D.

23. The method of claim 20, wherein each of steps (1) and (2) comprises metagenomics sequencing.

24. The method of claim 20, wherein each of steps (1) and (2) comprises polymerase chain reaction (PCR).

25. The method of claim 24, wherein the PCR is quantitative PCR (qPCR).

26. The method of claim 20, wherein the bacterial species is/are (i) Clostridium bartlettii, Haemophilus parainfluenzae, Escherichia coli, Lachnospiraceae bacterium 5_1_63FAA, Eubacterium ventriosum or, (ii) Clostridium bartlettii or, (iii) Haemophilus parainfluenzae or, (iv) Escherichia coli or, (v) Lachnospiraceae bacterium 5_1_63FAA or, (vi) Eubacterium ventriosum or, (vii) Clostridium bartlettii, Haemophilus parainfluenzae, Escherichia coli, Lachnospiraceae bacterium 5_1_63FAA or, (viii) Clostridium bartlettii, Haemophilus parainfluenzae, Escherichia coli, Eubacterium ventriosum or, (ix) Clostridium bartlettii, Haemophilus parainfluenzae, Lachnospiraceae bacterium 5_1_63FAA, Eubacterium ventriosum or, (x) Clostridium bartlettii, Escherichia coli, Lachnospiraceae bacterium 5_1_63FAA, Eubacterium ventriosum or, (xi) Haemophilus parainfluenzae, Escherichia coli, Lachnospiraceae bacterium 5_1_63FAA, Eubacterium ventriosum or, (xii) Clostridium bartlettii, Lachnospiraceae bacterium 5_1_63FAA, Eubacterium ventriosum or, (xiii) Clostridium bartlettii, Haemophilus parainfluenzae, Lachnospiraceae bacterium 5_1_63FAA or, (xiv) Haemophilus parainfluenzae, Escherichia coli, Lachnospiraceae bacterium 5_1_63FAA or, (xv) Clostridium bartlettii, Haemophilus parainfluenzae, Escherichia coli or, (xvi) Haemophilus parainfluenzae, Escherichia coli.

27. A kit for assessing risk of obesity and type 2 diabetes (T2D) in a subject or for assessing whether a subject has microbiome-dependent obesity and T2D, comprising reagents for detecting one or more of the bacterial species set forth in Tables 1-5.

28-31. (canceled)
